(19) United States
(12) Patent Application Publication
(10) Pub. No.: US 2004/0254545 A1
(43) Pub. Date: Dec. 16, 2004

(54) Method and apparatus for extending feeding tube longevity

(76) Inventors: Dean Loller Rider II, Tiburon, CA (US); Andrew Kees Roorda, San Francisco, CA (US)

Correspondence Address: Burt Magen, Vierra Magen Marcus Harmon & DeNiro LLP, Suite 540, 685 Market Street, San Francisco, CA, 94105, US

(21) Appl. No.: 10/462,365

(22) Filed: Jun. 16, 2003

(51) Int. Cl. A61M 25/00

(52) U.S. Cl. 604/265; 427/2.1

(57) ABSTRACT

The present invention is directed to a method and apparatus for a preventing or delaying the formation and proliferation of biofilm on a feeding tube and thereby extending tube longevity.

### BACKGROUND OF THE INVENTION

1. Field of the Invention

The present invention is directed to a method and apparatus for preventing or delaying the formation and proliferation of biofilm on a feeding tube and thereby extending tube longevity.

2. Description of the Related Art Gastrostomy tubes, skin-level devices (or buttons), and jejunostomy tubes are enteral feeding devices that enable the administration of nutritional solutions directly into the stomach or intestines. Such devices are manufactured by several companies and are commonly constructed of silicone, latex, or polyurethane (Solomon J M, Kirby D F. Percutaneous endoscopic gastrostomy: A matter of choice. Endoscopy Rev 1988;36:45). At present, silicone rubber is the most widely used material in the manufacture of percutaneous endoscopic gastrostomy (PEG) feeding tubes and PEG replacement tubes (Iber F L, Livak A, Patel M. Importance of fungus colonization in failure of silicone rubber percutaneous gastrostomy tubes (PEGs). Digestive Diseases and Sciences 1996;Jan;41(1):226-231). In brief, the PEG procedure involves the creation of a tract and subsequent placment of a feeding tube through the skin into the stomach utilizing both surgical and endoscopic methods. Enteral feeding as embodied by the aforementioned devices is indicated for patients who have an intact, functional gastrointestinal tract, but are unable to consume sufficient calories to meet metabolic demands (The Standards of Practice Committee of the American Society for Gastrointestinal Endoscopy. Role of PEG/PEJ in Enteral Feeding. ASGE Guidelines for Clinical Application. 1998).

PEG tubes have a limited lifespan and frequently need to be replaced. In a recent study of 363 PEG tubes placed over an eight year period, mean replacement time was shown to be 255 days (Koulentaki M, Reynolds N, Steinke D, Tait J, Baxter J, Vaidya K, Jayesakera A, Pennington C. Eight years' experience of gastrostomy tube management. Endoscopy 2002; Dec;34(12):941-5). It is estimated that over 250,000 gastrostomies (including approximately 4% in children) are performed annually in the United States (Gauderer M W L. Percutaneous endoscopic gastrostomy and the evolution of contemporary long-term enteral access. Clin Nutr 2002;21:103-110). This is part of an increasing trend. Gastrostomies placed in hospitalized patients aged 65 years or older in the United States increased from 61,000 in 1988 to 121,000 in 1995 (Graves E J. Detailed diagnoses and procedures: National Hospital Discharge Survey, 1988. Vital Health Stat 13 1991;107:116; Graves E J, Gillum B S. Detailed diagnoses and procedures: National Hospital Discharge Survey, 1995. Vital Health Stat 13. 1997;130:124). Over the course of the next fifty years, the share of the elderly (defined as those aged 65 years and above) is expected to climb from 6.9 percent of the total population to 15.6 percent [Medium Variant Projections of the United Nations (UN) 2001]. Consequently, the number of gastrostomy tubes placed annually will likely further increase.

One of the principal causes of device failure for gastrostomy tubes is fungal colonization. One study demonstrated a tube failure rate secondary to fungal colonization of 37% of silicone tubes in place for 250 days and in 70% of tubes in place for 450 days (Iber F L, Livak A, Patel M. Importance of fungus colonization in failure of silicone rubber percutaneous gastrostomy tubes (PEGs). Digestive Diseases and Sciences 1996 January;41(1):226-231). The following were cited as reasons why the authors believed fungus to be responsible for tube failure: no deterioration was observed in the tubes completely free of fungal colonization, all dilatation and brittleness was confined to portions of the tube that were heavily colonized by fungi, and no abnormalities were noted in uncolonized portions of the feeding tubes.

Examination of feeding tubes that have failed secondary to fungal colonization has demonstrated dilatation, brittleness, obstruction, nodularity, tears, loss of elasticity, and color changes (opacification and/or dark discoloration) [Marcuard S P, Finley J L, MacDonald K G. Large-bore feeding tube occlusion by yeast colonies. Journal of Parenteral and Enteral Nutrition 1993;17(2):187-190; Iber F L, Livak A, Patel M. Importance of fungus colonization in failure of silicone rubber percutaneous gastrostomy tubes (PEGs). Digestive Diseases and Sciences 1996 January;41(1):226-231]. Iber et al. emphasized that the majority of cases of failed tubes had two or more of the above types of abnormalities. Fungal colonization of feeding tubes is also suspected of causing the following: tube breaking or fracturing, kinking, loss of resilience, and variations in external diameter (Marcuard S P, Finley J L, MacDonald K G. Large-bore feeding tube occlusion by yeast colonies. Journal of Parenteral and Enteral Nutrition 1993;17(2):187-190; Gottlieb K, DeMeo M, Borton P, Mobarhan S. Gastrostomy tube deterioration and fungal colonization. American Journal of Gastroenterology 1992 November;87(11):1683; Gottlieb K, Iber F L, Lavak A, Leya J, Mobarhan S. Oral Candida Colonizes the Stomach and Gastrostomy Feeding Tubes. Journal of Parenteral and Enteral Nutrition 1994; 18(3):264-267).

A total loss of elasticity from fungal colonization can occur as early as 150 days following initial feeding tube placement. Also, dense yeast colonies can penetrate approximately forty percent of the tube wall by three to four months (Marcuard S P, Finley J L, MacDonald K G. Large-bore feeding tube occlusion by yeast colonies. Journal of Parenteral and Enteral Nutrition 1993;17(2):187-190). Another study corroborated this finding by showing that on frozen section, fungi had invaded the wall of the tubing (Iber F L, Livak A, Patel M. Importance of fungus colonization in failure of silicone rubber percutaneous gastrostomy tubes (PEGs). Digestive Diseases and Sciences 1996 January;41(1):226-231).

Biofilm colonization of gastrostomy tubes may also play a significant role in the formation of granulation tissue which can occlude the tube lumen and lead to device failure (Dautle M P, Wilkinson T R, Gauderer M W. Isolation and identification of biofilm microorganisms from silicone gastrostomy devices. J Pediatr Surg 2003 February;38(2):216-220).

Consequently, feeding tubes have to be frequently replaced at great cost, inconvenience, and discomfort to the patient (Sartori S, Trevisani L, Nielsen I, Tassinari D, Ceccotti P, Abbasciano V. Longevity of silicone and polyurethane catheters in long-term enteral feeding via percutaneous endoscopic gastrostomy. Aliment Pharm Ther 2003 March;17(6):853-6; Gottlieb K, Leya J, Kruss D M, Mobarhan S, Iber F L. Intraluminal fungal colonization of gastrostomy tubes. Gastrointest Endosc 1993;39:413-415; Koulentaki M, Reynolds N, Steinke D, Tait J, Baxter J, Vaidya K, Jayesakera A, Pennington C. Eight years' experience of gastrostomy tube management. Endoscopy 2002 December;34(12):941-5).

More often than not feeding tubes are the only means of administering medications, fluid, and nutrition. Since feeding tube placement often requires the assembly of a skilled team including a gastroenterologist, surgeon, anesthesiologist, and endoscopic nurse, prompt tube replacement may not be readily available (Rider D L, Rider J A, Roorda A K. PEG: A Safe Procedure In The Elderly: Including The Oldest Old, Practical Gastroenterology 2002 August;XXVI(8):38-44.). If vital nutrients are not placed in a timely fashion the patient the patient is at risk for morbidity and possibly mortality.

Additionally, the presence of fungal colonies on the feeding tube places the patient at risk for complications such as Candida peritonitis and Candida cellulitis and possibly even fungemia (Murugasu B, Conley S B, Lemire J M, et al. Fungal peritonitis in children treated with peritoneal dialysis and gastrostomy feeding. Pediatr Nephrol 1991;5:620-1; Patel A S, DeRidder P H, Alexander T J, Veneri R J, Lauter C B. Candida cellulitis: a complication of percutaneous endoscopic gastrostomy. Gastrointestinal Endoscopy 1989;35(6):571-572; Komshian S V, Uwaydah A K, Sobel J D, Crane L R. Fungemia caused by Candida species and Torulopsis glabrata in the hospitalized patient: frequency, characteristics, and evaluation of factors influencing outcome. Rev Infect Dis 1989;11:379-90). Several authors believe that candidal overgrowth predisposes to fungemia (Stone H H, Geheber C E, Kolb L D, et al. Alimentary tract colonization by Candida albicans. J Surg Res 1973;14:273-276; Kennedy M J, Volz P A. Ecology of Candida albicans gut colonization: Inhibition of Candida adhesion, colonization, and dissemination by bacterial antagonism. Infect Immun 1985;49:654-666). One study places the attributable mortality of candidemia at 38 percent (Wey S B, Mori M, Pfaller M A, Woolson R F, Wenzel R P. Hospital acquired candidemia: the attributable mortality and excess length of stay. Arch Intern Med 1988;148:2642-5).

A recent article showed that bacterial microorganisms found a niche under protective outer layers of fungi (Dautle M P, Wilkinson T R, Gauderer M W. Isolation and identification of biofilm microorganisms from silicone gastrostomy devices. J Pediatr Surg 2003 February;38(2):216-20). A biofilm is a community of microorganisms attached to a solid surface. Such surfaces include feeding tubes, catheters, medical implants, wound dressings, or other types of medical devices. Once established, biofilm microorganisms are impossible to treat with antimicrobial agents and detachment from the device may result in infection (Donlan R M. Biofilms and device-associated infections. Emerging Infectious Diseases 2001. 7(2):277-281). Biofilm microorganisms are known to exhibit increased resistance to antibiotics (Costerton J W, Lewandowski Z: The biofilm lifestyle. Adv Dent Res 1997;11:192-195). Candida albicans biofilm formation has also been shown to be positively correlated with cell surface hydrophobicity (Li X, Yan Z, Xu J. Quantitative variation of biofilms among strains in natural populations of Candida albicans. Microbiology 2003 February; 149(Pt 2):353-62).

Several fungal organisms have been implicated in feeding tube failure. It is likely that these fungi colonize the tube as a biofilm at or soon after initial placement. Theoretically, this colonization can occur at any anatomical point between insertion of the tube into the oral cavity and extrusion through the stoma. Colonization can also possibly occur prior to or at any point in time after placement. Recovery of fungal or bacterial organisms appears greater from the lumen of gastrostomy tubes as opposed to the exterior surface (Gottlieb K, Iber F L, Lavak A, Leya J, Mobarhan S. Oral Candida Colonizes the Stomach and Gastrostomy Feeding Tubes. Journal of Parenteral and Enteral Nutrition 1994;18(3):264-267).

It has been hypothesized that seeding of the gastrostomy site occurs during passage of the tube through a potentially infected oropharynx (Patel A S, DeRidder P H, Alexander T J, Veneri R J, Lauter C B. Candida cellulitis: a complication of percutaneous endoscopic gastrostomy. Gastrointest Endosc 1989;35:571-2). Gottlieb et al. also hypothesized that luminal surface of gastrostomy tubes becomes colonized as the bumper is pulled through the oral cavity (Gottlieb K, Leya J, Kruss D M, Mobarhan S, Iber F L. Intraluminal fungal colonization of gastrostomy tubes. Gastrointest Endosc 1993;39:413-415). They speculate that the bumper subsequently acts as a bridgehead for further advancement of microorganisms into the lumen. Gottlieb et al. provided further support to this theory by demonstrating that species isolated from the oral cavity, the stomach, and later the gastrostomy tube were identical in most cases (Gottlieb K, Iber F L, Lavak A, Leya J, Mobarhan S. Oral Candida Colonizes the Stomach and Gastrostomy Feeding Tubes. Journal of Parenteral and Enteral Nutrition 1994;18(3):264-267).

Esophageal candidiasis, if present, could colonize the feeding tube as it is passed from the oropharynx into the stomach. The stomach itself could allow entry of Candida tropicalis, which is more commonly found in the lower gastrointestinal tract than the oral cavity (Edwards J E. Candida species. In: Mandell G L, Douglas R G, Bennett J E, eds. Principles and practice of infectious diseases. New York: Churchill Livingstone, 1990:1943-58). The fact that fungal growth in feeding tubes is often heaviest adjacent to the bumper lends support to this theory (Gottlieb K, Iber F L, Lavak A, Leya J, Mobarhan S. Oral Candida Colonizes the Stomach and Gastrostomy Feeding Tubes. Journal of Parenteral and Enteral Nutrition 1994; 18(3):264-267). Another study showed that fungal colonization of PEG tubes was always the portion of the tube most proximal to the patient, extending to a maximum of 11 cm in the most extreme case (Iber F L, Livak A, Patel M. Importance of fungus colonization in failure of silicone rubber percutaneous gastrostomy tubes (PEGs). Digestive Diseases and Sciences 1996 January;41(1):226-231).

One author has suggested that Candida is an external contaminant that after first colonizing the gastrostomy tube, might secondarily infect the patient (Gillanders I A, Davda N S, Danesh B J. Candida albicans infection complicating percutaneous endoscopic gastrostomy (Letter). Endoscopy 1992;24:733).

Fungal colonization of the stomach has been associated with conditions of increased gastric pH such as H₂ blocker therapy (Minoli G, Terruzzi V, Ferrara, et al. A prospective study of relationships between benign gastric ulcer, Candida and medical treatment. Am J Gastroenterol 1984;79:95-7). The elderly, through the normal aging process, have decreased gastric acid secretion, or achlorhydria. Since the elderly comprise the predominant patient population receiving feeding tubes, it is likely that their increased stomach pH places them at greater risk of colonization of the feeding tube as it is passed through the stomach. Malnutrition, one of the manifestations of failure to thrive, is one of the main indications for feeding tube placement. Malnutrition may be one of the most important risk factors for colonization (Odds F C. Ecology and epidemiology of Candida species. Zentrabl Bakt Hyg A 1984;257:207-212; Kennedy R J, Rogers A I, Yancey R J. An anaerobic continuous-flow culture model of interactions between intestinal microflora and Candida albicans. Mycopathologia 1988;103:141-143). The well-documented suppression of cellular immunity in malnutrition is a likely explanation (Raymond H P, Shou J, Kelly C J, et al. Immunosuppressive mechanisms in protein-calorie nutrition. Surgery 1991;110:311-317). The consequential proliferation of fungal organisms occurs either directly or through the disruption of the indigenous microflora that normally act to suppress fungi (Gottlieb K, Iber F L, Lavak A, Leya J, Mobarhan S. Oral Candida Colonizes the Stomach and Gastrostomy Feeding Tubes. Journal of Parenteral and Enteral Nutrition 1994;18(3):264-267). One study demonstrated that 65% of patients had oral and/or stomach colonization of Candida species at the time of initial PEG placement (Gottlieb K, Iber F L, Lavak A, Leya J, Mobarhan S. Oral Candida Colonizes the Stomach and Gastrostomy Feeding Tubes. Journal of Parenteral and Enteral Nutrition 1994;18(3):264-267).

A significantly higher incidence of Candida species has been found in the gastric and small-intestinal aspirates of malnourished children when compared to normal well-nourished controls (Gracey M, Stone D E, Suharjono S H, Sunoto I T. Isolation of Candida species from the gastrointestinal tract in malnourished children. Am J Clin Nutr 1974;27:345-9). With few exceptions, Candida from the patients own endogenous microflora is the main cause of human Candida infections and presumably, the cause of Candida colonization of prostheses and devices (Odds F C. Ecology and epidemiology of Candida species. Zbl Bakt Hyg A 1984;257:207-12).

One of the earliest studies described a failed tube that upon subsequent culture confirmed the presence of Candida tropicalis, Candida albicans, Torulopsis glabrata, Engyodontium album, and Wangiella dermatitides (Gottlieb K, DeMeo M, Borton P, Mobarhan S. Gastrostomy tube deterioration and fungal colonization. American Journal of Gastroenterology 1992 November;87(11):1683). Another study of feeding tubes tubes that failed secondarily to fungal colonization also implicated Candida (Iber F L, Livak A, Patel M. Importance of fungus colonization in failure of silicone rubber percutaneous gastrostomy tubes (PEGs). Digestive diseases and sciences 1996 January;41(1):226-231). Candida albicans was also implicated in PEG tube failure in a recent study (Koulentaki M, Reynolds N, Steinke D, Tait J, Baxter J, Vaidya K, Jayesakera A, Pennington C. Eight years' experience of gastrostomy tube management. Endoscopy 2002 December;34(12):941-5). Wangiella can lead to localized skin and subcutaneous infections. Endocarditis has also been reported (Vartian C V, Shleas D M, Padhve A A, et al. Wangiella dermatitidis endocarditis in an intravenous drug user. Am J Med 1985;78:703-7). E. album has been implicated in aortic valve endocarditis, keratitis, brain abscess, and eczema vesiculosum (Augustinsky J, Kammeyer P, Husain A, et al. Engyodontium album endocarditis. J Clin Microbiol 1990;28:1479-81). Stomal wound dressings over the gastrostomy site are another suspected cause of fungal colonization and superficial infection (Patel A S, DeRidder P H, Alexander T J, Veneri R J, Lauter C B. Candida cellulitis: a complication of percutaneous endoscopic gastrostomy. Gastrointest Endosc 1989;35:571-2). While not explicitly implicated in feeding tube deterioration, Candida krusei has been cited in the literature as having colonized feeding tubes (Gottlieb K, Leya J, Kruss D M, Mobarhan S, Iber F L. Intraluminal fungal colonization of gastrostomy tubes. Gastrointest Endosc 1993;39:413-415; Marcuard S P, Finley J L, MacDonald K G. Large-bore feeding tube occlusion by yeast colonies. Journal of Parenteral and Enteral Nutrition 1993;17(2):187-190).

It is plausible that a variety of fungal organisms found on the surface of feeding tubes play a role in their deterioration. Many fungi can utilize crude oil and therefore could degrade petroleum-based polymers (Davies J S, Westlake D W. Crude oil utilization by fungi. Can J Microbiol 1979;25:146-56). Also, the utilization of intermediate chain length hydrocarbons has been reported for several species of the genera Torulopsis, Candida, and Aspergillus (Klug M J, Markovetz A J. Utilization of aliphatic hydrocarbons by microorganisms. Adv Microbiol Physiol 1971;5:1-43). It appears that both synthetic and semisynthetic complex polymers are vulnerable to corrosion by microbial organisms. The growth properties of selected fungi on polyvinyl chloride film have been studied and it was determined that all fungi use epoxidized oil, a plasticizer-stabilizer, as a carbon source (Roberts W T, Davidson P M. Growth characteristics of selected fungi on polyvinyl chloride film. Appl Environ Microbiol 1986;51:673-6). Gottlieb et al. hypothesized that the synthetic polymers of PEGs (mostly silicone and some polyurethane) are vulnerable to attack by fungi (Gottlieb K, Leya J, Kruss D M, Mobarhan S, Iber F L. Intraluminal fungal colonization of gastrostomy tubes. Gastrointest Endosc 1993;39:413-415). Certain fungal organisms can flourish on the feeding tube substrate provided the presence of a warm and moist substrate. 37° C. temperature, high humidity, and the regular provision of fresh culture medium make feeding tubes the ideal incubator. One study commented that gastrostomy tubes could act as portable incubators where fungi or bacteria not only survive but thrive and multiply, spilling in huge numbers into the GI tract whenever feedings are flushed through the tube (Gottlieb K, Iber F L, Lavak A, Leya J, Mobarhan S. Oral Candida Colonizes the Stomach and Gastrostomy Feeding Tubes. Journal of Parenteral and Enteral Nutrition 1994;18(3):264-267). The implication of this includes the alteration of normal gastric flora and the subsequent overwhelming of immune systems that already are compromised in many instances. Once established, their niche within the feeding tube has been impregnable by host cellular and humoral immune defense mechanisms and antimicrobials.

Candida tropicalis possesses an alkane-inducible cytochrome P-450, which enables it to use alkanes as a carbon source (Sanglard D, Loper J C. Characterization of the alkane-inducible cytochrome P450 (P450alk) gene from the yeast Candida tropicalis: identification of a new P450 gene family. Gene 1989;76:121-36). It also produces biosurfactants which emulsify hydrocarbons (Singh M, Desai J D. Hydrocarbon emulsification by Candida tropicalis and Debaryomyces polymorphus. Indian J Exp Biology 1989;27:224-6). Polymer additives such as plasticizers (polymer softeners) are incorporated into feeding tubes during the manufacturing process. Their presence may explain why the internal bumpers of PEG tubes, which are soft at first, harden after several months (Foutch P G, Woods C A, Talbert G A, Sanowski R A. A critical analysis of the Sacks-Vine gastrostomy tube: a review of 120 consecutive procedures. Am J Gastroenterol 1988;83:812-5). Elimination of the plasticizer by fungal metabolism has been shown to make plastic film brittle. This increases tensile strength and decreases elongation potential, the net effect being that the films become stiff (Roberts W T, Davidson P M. Growth characteristics of selected fungi on polyvinyl chloride film. Appl Environ Microbiol 1986;51:673-6). Bacterial-fungal synergism is within the realm of possibility as bacterial organisms, including Pseudomonas aeruginosa, have been implicated in the degradation of synthetic polymers (Toepfer C T, Kanz E. Mutual relations between plastic materials and bacteria [German]. Zbl Bakt Hyg B 1976;163:540-55). The following bacteria are known to colonize gastrostomy tubes: Actinomyces pyogenes, α streptococci, Bacillus brevis, Bacillus licheniformis, Bacillus megaterium, Bacillus pumilus, Bacillus subtilis, Corynebacterium aquaticum, Corynebacterium pseudodiphtheriticum, Enterobacter cloacae, Enterococcus durans, Enterococcus faecalis, Enterococcus faecium, Enterococcus hirae, Escherichia coli, Klebsiella pneumoniae ssp pneumoniae, Lactobacillus plantarum, Lactobacillus species, Micrococcus kristinae, Micrococcus luteus, Micrococcus sedentarius, Proteus mirabilis, Proteus species, Pseudomonas aeruginosa, Serratia species, Staphylococcus aureus, Staphylococcus epidermidis, Staphylococcus intermedius, Staphylococcus saprophyticus, Xanthomonas maltophila, Yersinia enterocolitica group. (Gottlieb K, Leya J, Kruss D M, Mobarhan S, Iber F L. Intraluminal fungal colonization of gastrostomy tubes. Gastrointest Endosc 1993;39:413-415; Marcuard S P, Finley J L, MacDonald K G. Large-bore feeding tube occlusion by yeast colonies. Journal of Parenteral and Enteral Nutrition 1993;17(2):187-190; Dautle M P, Ulrich R L, Hughes T A. Typing and subtyping of 83 clinical isolates purified from surgically implanted silicone feeding tubes by random amplified polymorphic DNA amplification. Journal of Clinical Microbiology 2002;40(2):414-421; Dautle M P, Wilkinson T R, Gauderer M W. Isolation and identification of biofilm microorganisms from silicone gastrostomy devices. J Pediatr Surg 2003February;38(2):216-220).

Since tubes fail they must be frequently replaced. This puts the patient at risk of unique complications associated with replacement. The following complications associated with PEG replacement have been reported in the literature: duodenal obstruction, death, bleeding gastric ulcer, peritonitis, gastrocolic fistula, gastric outlet obstruction, small intestinal perforation, esophageal perforation, and hemoperitoneum (Strock P, Baroudi A, Sounni A, Fort E, Laurin C, Sapey T. Duodenal obstruction by balloon of replacement tube of percutaneous endoscopic gastrostomy. Gastroenterol Clin Biol 2002 June-July;26(6-7):640-1; Platt M S, Roe D C. Complications following insertion and replacement of percutaneous endoscopic gastrostomy (PEG) tubes. J Forensic Sci 2000 July;45(4):833-5; Delatore J, Boylan J J. Bleeding gastric ulcer: a complication from gastrostomy tube replacement. Gastrointest Endosc 2000 April;51(4 Pt 1):482-4; Shahbani D K, Goldberg R. Peritonitis after gastrostomy tube replacement in the emergency department. J Emerg Med 2000 January;18(1):45-6; Hudziak H, Loudu P, Bronowicki J P, Claviere C, Chone L, Bigard M A. Diarrhea following the replacement of percutaneous endoscopic gastrostomy tube: to think of gastrocolic fistula. Gastroenterol Clin Biol 1996;20(12):1139-40; Walsh M J, Clement D J. Replacement gastrostomy tube as a cause of gastric outlet obstruction. Gastrointest Endosc 1990 November-December;36(6):640.; Wilson W C, Zenone E A, Spector H. Small intestinal perforation following replacement of a percutaneous endoscopic gastrostomy tube. Gastrointest Endosc 1990 January-February;36(1):62-3; Kenigsberg K, Levenbrown J. Esophageal perforation secondary to gastrostomy tube replacement. J Pediatr Surg 1986 November;21(11):946-7; Tan Y M, Abdullah M, Goh K L. Hemoperitoneum after accidental dislodgement and subsequent replacement of PEG tube. Gastrointest Endosc 2001 May;53(6):671-3; Spiegelman G, Goldberg R I. Gastric ulceration following PEG replacement. Gastrointest Endosc 1992 May-June;38(3):397-8).

Thereafter, researchers attempted methods of extending feeding tube longevity. Despite injecting a nystatin suspension (500,000 units/5 mL) into the tube during the off cycle to fill the entire tube lumen, Marcuard et al. were unsuccessful in attempting to clear a feeding tube colonized with Candida (Marcuard S P, Finley J L, MacDonald K G. Large-bore feeding tube occlusion by yeast colonies. Journal of Parenteral and Enteral Nutrition 1993;17(2):187-190). A second attempt was made by Marcuard et al. using a solution of amphotericin B (1 mg/10 mL). This was applied for one week in a similar fashion as the nystatin but proved unsuccessful. Due to intermittent episodes of occlusion this tube was removed. Segments of the tube were incubated in vitro overnight in similar nystatin and amphotericin B solutions. On the following day attempts were made by Marcuard et al. to clear the yeast crust from the inner surface of the tube using an endoscopic brush. This also proved unsuccessful. Other researchers using similar methods of rubbing or washing fungus-infested tubes also failed in their efforts to dislodge the colonies (Iber F L, Livak A, Patel M. Importance of fungus colonization in failure of silicone rubber percutaneous gastrostomy tubes (PEGs). Digestive Diseases and Sciences 1996 January;41(1):226-231).

Prophylactic measures to obviate the problem of fungal colonization of feeding tubes have also apparently failed. One such approach involved pre-procedural preparation of the oropharynx with 1% neomycin (Grief J M, Ragland J J, Ochsner M G, Riding R. Fatal necrotizing fasciitis complicating percutaneous endoscopic gastrostomy. Gastrointest Endosc 1986;32:292-4).

One group of researchers proposed stopping and rescheduling the procedure if esophageal candidiasis is discovered during endoscopy at the beginning of the gastrostomy tube placement procedure (Patel A S, DeRidder P H, Alexander T J, Veneri R J, Lauter C B. Candida cellulitis: a complication of percutaneous endoscopic gastrostomy. Gastrointest Endosc 1989;35:571-2.). Again, this is unrealistic in that it increases cost and patient inconvenience. The patient also may not be able to mount an adequate immune response to the infection since in many cases they are already suffering from nutritional deficiency at the time of placement.

Another study suggested that the use of polyurethane tubes may offer a solution to the problems posed by fungal colonization (Iber F L, Livak A, Patel M. Importance of fungus colonization in failure of silicone rubber percutaneous gastrostomy tubes (PEGs). Digestive Diseases and Sciences 1996 January;41(1):226-231). However, this data is unreliable since many of these tubes were placed surgically, thus bypassing any oropharyngeal/esophageal fungal flora. While surgical placement of gastrostomy tubes was common at one time, it is no longer the procedure of choice. As with any abdominal surgery there were a host of complications. Also, a recent study showed that failure rates were nearly identical for silicone and polyurethane tubes (Van del Hazel S J, Mulder C J J, Den Hartog G, Thies J E, Westhof W. A randomized trial of polyurethane and silicone percutaneous endoscopic gastrostomy catheters. Aliment Pharmacol Ther 2000;14:1273-7). Also, studies investigating fungal colonization of polyurethane catheters are lacking in the medical literature (Sartori S, Trevisani L, Nielsen I, Tassinari D, Ceccotti P, Abbasciano V. Longevity of silicone and polyurethane catheters in long-term enteral feeding via percutaneous endoscopic gastrostomy. Aliment Pharm Ther 2003 March;17(6):853-6).

Koulentaki et al. advocated the use of more durable materials in the manufacture of gastrostomy tubes, but they did not provides specific suggestions of these materials (Koulentaki M, Reynolds N, Steinke D, Tait J, Baxter J, Vaidya K, Jayesakera A, Pennington C. Eight years' experience of gastrostomy tube management. Endoscopy 2002 December;34(12):941-5).

U.S. Pat. No. No. 6,165,168 claims that it is an improvement in that it extends the indwelling longevity of the device. However, it fails to mention the precise mechanism. U.S. Pat. No. No. 6,165,168 infers that the longevity is somehow extended by preventing backflow leakage. Reaction by the body to such devices is defined by U.S. Pat. No. 6,165,168 as consisting of an inflammation or an infection. While backflow leakage may contribute to feeding tube wear and tear, its effect is minimal compared to fungal colonization. While U.S. Pat. No. 6,165,168 may somewhat extend longevity, it does not extend longevity to the extent that the present invention does since it does not address the issue of fungal colonization.

Therefore, what is needed is an improvement to feeding tubes that extends feeding tube longevity.

### SUMMARY OF THE INVENTION

The present invention, roughly described, pertains to extending the longevity of feeding tubes. More specifically, it pertains to extending the longevity of feeding tubes by utilizing one or more anti-biofilm mechanisms. The objective of an anti-biofilm mechanism is to inhibit and/or delay the formation and/or proliferation of fungal/and or bacterial biofilm. An anti-biofilm mechanism may be direct (biofilmacidal) or indirect (biofilmostatic). The term biofilmacidal means destructive or lethal to biofilm. The term biofilmostatic means inhibiting growth or multiplication of biofilm. It is believed that the terms biofilmacidal and biofimostatic are novel with respect to the prior art.

One example of an anti-biofilm mechanism is surface treatment and/or surface modification. An example of a surface treatment and/or surface modification of a feeding tube is surface functionalization. Surface functionalization of a feeding tube involves insertion of a functional group onto the surface in order to improve its wettability, sealability, its resistance to glazing, or its adhesion to other polymers or metals. Surface functionalization maintains the desirable bulk properties of the feeding tube. Surface functionalization of a feeding tube can also be used to improve barrier characteristics of polymers and to impart polymers with antifungal and/or antibacterial properties.

Another example of a surface treatment and/or surface modification of feeding tubes is surface cleaning and/or etching. This process involves cleaning and/or etching feeding tube surfaces by removing unwanted materials and contaminants from polymer surface layers. Such unwanted materials and contaminants can act as a nidus for biofilm formation and/or proliferation.

Another example of a surface treatment and/or surface modification of feeding tubes is surface deposition. This process involves the deposition of thin layers of coatings on polymer substrate surfaces. Examples of coatings include antifungals, antibacterials, antiseptics, disinfectants, metals, metallic ions, metal alloys, metals conjugated with another anti-biofilm mechanism, therapeutic agents that block gene expression, therapeutic agents that inhibit and/or delay the formation and/or proliferation of granulation tissue, and a therapeutic agents that inhibit and/or delay the formation and/or proliferation of inorganic salts.

Comprehensive descriptions of the art of traditional surface treatment and/or surface modification can be found in A Guide to Metal and Plastic Finishing (Maroney, Marion L.; 1991), Handbook of Semiconductor Electrodeposition (Applied Physics, 5) (Pandey, R. K., et. al.; 1996), Surface Finishing Systems: Metal and Non-Metal Finishing Handbook-Guide (Rudzki, George J.; 1984), and Materials and Processes for Surface and Interface Engineering (NATO Asi Series. Series E, Applied Sciences, 115) (Pauleau, Ives (Editor); 1995); herein incorporated by reference.

An anti-biofilm mechanism may or may not involve a surface treatment and/or modification. An anti-biofilm agent can be a therapeutic agent. Therapeutic agents include antiseptics, disinfectants, antifungals, antibiotics, metals, molecules that disrupt steps of the biofilm lifecycle, molecules that block gene expression, molecules that block the formation of granulation tissue, and molecules that block the formation of inorganic salts. Other suitable therapeutic agents can also be used. Such therapeutic agents can be applied to a feeding tube as a surface treatment and/or modification, incorporated into reservoirs with or without an overlying surface treatment, incorporated into the constituent body of the feeding tube with or without an overlying surface treatment, or by other means.

Antiseptics are generally defined as compounds that kill or inhibit the growth of microorganisms on skin or living tissue. Antiseptics include, but are not limited to, alcohols, chlorhexedine, iodophors and dilute hydrogen peroxide. Other antiseptics include guanidium compounds, biguanides, bipyridines, phenoxide antiseptics, alkyl oxides, aryl oxides, thiols, aliphatic amines, aromatic amines and halides such as F⁻, Br⁻, and I⁻. Some examples of guanidium compounds that may be used include chlorhexedine, alexidine, and hexamidine. One example of a bipyridine compound that can be used to synthesize the antiseptics of the invention is octenidine. Examples of phenoxide antiseptics used include colofoctol, chloroxylenol, and triclosan.

Disinfectants are compounds that eliminate pathogenic microorganisms from inanimate surfaces and are generally more toxic, and hence more effective, than antiseptics. Representative disinfectants include, but are not limited to, formaldehyde, quaternary ammonium compounds, phenolics, bleach and concentrated hydrogen peroxide. Antibiotics and antifungals are compounds that can be administered systemically to living hosts and exhibit selected toxicity. These compounds interfere with selected biochemical pathways of microorganisms at concentrations that do not harm the host. Examples of antifungals that can be used as a therapeutic agent in an extended-longevity feeding tube include echinocandins or glucan synthase inhibitors (caspofungin, micafungin, anidulafungin), allylamines and other non-azole ergosterol biosynthesis inhibitors (amorolfine, butenafine, naftifine, terbinafine), antimetabolites (flucytosine), azoles (fluconazole, itraconazole, ketoconazole, posaconazole, ravuconazole, voriconazole, clotrimazole, econazole, miconazole, oxiconazole, sulconazole, terconazole, and tioconazole), chitin synthase inhibitors (nikkomycin Z), polyenes (amphotericin B (AmB), AmB lipid complex, AmB colloidal dispersion, liposomal AmB, AmB oral suspension, liposomal nystatin, topical nystatin, pimaricin), griseofulvin, ciclopiroxolamine, rM-CSF. Other suitable antifungals can also be used. Examples of antibiotics that can be used as a therapeutic agent in an extended-longevity feeding tube include aminoglycosides, β-lactams, cephalosporins, macrolides and combinations, penicillins, quinolones, sulfonamides and combinations, tetracyclines, clindamycin, colistimethate, quinupristin/dalfopristin, vancomycin, linezolid, ABT-773, evernimicin, ciprofloxacin, MBI 226, lomefloxacin, ertapenem, iseganan, ramoplanin, gemifloxacin mesylate, amoxicillin/clavulanate, moxifloxacin, daptomycin, GAR-936, telithromycin, clarithromycin, AZD2563, peperacillin/tazobactam, dalbavancin, des 6-fluoroquinolone, oritavancin, BB-83698, and BAL5788. Other suitable antibiotics can also be used.

The fundamental difference between antiseptics, disinfectants and antibiotics/antifungals is the ability of microorganisms to develop resistance to antibiotics/antifungals. The characteristics that make antiseptics and disinfectants so effective generally preclude the development of resistant microorganisms. However, some disinfectants can be unsuitable for use on living tissues and many antiseptics are primarily limited to localized, generally topical, applications. Consequently, most antimicrobial prophylactic and therapeutic regimens have traditionally relied on antibiotics/antifungals.

The antimicrobial effects of metallic ions such as Ag, Au, Pt, Pd, Ir (i.e. the noble metals), Cu, Sn, Sb, Bi and Zn are known (see Morton, H. E., Pseudomonas in Disinfection, Sterilization and Preservation, ed. S. S. Block, Lea and Febiger, 1977 and Grier, N., Silver and Its Compounds in Disinfection, Sterilization and Preservation, ed. S. S. Block, Lea and Febiger, 1977). A microbe is defined as a minute living organism, a microphyte or microzoon; applied especially to those minute forms of life which are capable of causing disease in animals, including bacteria, protozoa, and fungi (Dorland's Illustrated Medical Dictionary, Twenty-fifth edition, Saunders).

Molecules can be created to block the expression of genes that have been deemed pivotal in the biofim lifecycle. Examples of such genes include FLO11 (required for fungal biofilm formation), Efg1, Deltaefg1, Deltacph1/Deltaefg11, ALS (agglutinin-like), CDR (efflux pump), MDR (efflux pump). Such molecules can be applied as part of a therapeutic agent to a feeding tube.

An example of a molecule that interferes with steps of the biofilm lifecycle is farnesol. Farnesol has the chemical formula C₁₅H₂₆O. A feeding tube utilizing farnesol as a therapeutic agent inhibits filamentation in Candida albicans.

An example of a molecule that inhibits and/or delays formation and/or proliferation of feeding tube granulation tissue is albumin.

Other therapeutic agents may be utilized. In one embodiment, the considerations include (1) wherein the substance contains molecules that block or disrupt fungal and bacterial arrangement or attachment; (2) wherein the substance interferes with bacterial and fungal extracellular matrix formation; (3) wherein the substance delivers signal blockers to threatened areas to abort fungal or bacterial biofilm formation; (4) wherein the substance delivers multiple antifungals, antibiotics, or disinfectants to undermine the varied survival strategies of biofilm cells; (5) wherein the substance induces fungal and bacterial cells to detach, then targets them with antibiotics, antifungals, disinfectants, or antibodies.

An extended longevity feeding tube is different from antimicrobial impregnated central venous catheters or other catheters that are presently on the market. For example, a central venous catheter is a long fine catheter introduced via a large vein into the superior vena cava or right atrium for administration of parenteral fluids or medications or for measurement of central venous pressure. A feeding tube is a hollow cylindrical instrument for introducing high-caloric enteral foods, fluids or medications into the stomach. Parenteral nutrition bypasses the alimentary canal. Enteral nutrition does not. Parenteral nutrition involves infusion through a catheter via other routes such as intravenous, subcutaneous, intramuscular, etc. Enteral nutrition is nutrition provided through the gastrointestinal tract, taken by mouth, or provided through a tube that delivers nutrients directly into the stomach or into the small intestine.

One embodiment of preparing an extended-longevity feeding tube involves applying one or more therapeutic agents to the feeding tube via surface treatments. Feeding tube surface treatments include but are not limited to the following: dipping, spraying, solvent casting techniques, matrix loading, drug-polymer conjugates, vacuum-deposition techniques, diffusion (nitriding, carburizing), laser processes, plasma processes, chemical plating, grafting, bonding, bombardment with energetic particles (as in plasma immersion or ion implantation), gamma radiation, glow discharge techniques, biomimetic techniques, flame treatment processes, and ultraviolet processes.

Another embodiment of preparing an extended-longevity feeding tube involves the creation of one or more reservoirs containing one or more therapeutic agents underlying a layer that has been surface treated. An example of a surface treatment is a coating or a membrane of biocompatible material. This could be applied over the reservoirs which would control the diffusion of the drug from the reservoirs to the interior/exterior of the feeding tube. One advantage of this system is that the properties of the coating can be optimized for achieving superior biocompatibility and adhesion properties, without the additional requirement of being able to load and release the drug. The size, shape, position, and number of reservoirs can be used to control the amount of drug, and therefore the dose delivered to the internal and/or external surface of the feeding tube.

An additional embodiment of preparing an extended-longevity feeding tube includes a polymer having both bulk distributed therapeutic agent and an overlying surface treatment with or without a therapeutic agent. This embodiment can produce a dual extended-longevity activity feeding tube. The surface coating can provide a readily available and rapid release of a therapeutic agent. The bulk distributed therapeutic agent, due to the hydrophilic nature of the polymer, migrates slowly to the surface when the feeding tube is in contact with a fluid and produces extended-longevity activity of long duration.

In addition to the aforementioned methods of preparing an extend-longevity feeding tube, methods are provided for placing and using an extended-longevity feeding tube.

In summary, this invention provides a method and apparatus for extending feeding tube longevity. One embodiment of a feeding apparatus comprises a feeding tube. The feeding tube includes one or more surfaces having one or more anti-biofilm mechanisms. Another embodiment of a feeding apparatus comprises a feeding tube. The feeding tube includes one or more reservoirs. The reservoirs include one or more anti-biofilm mechanisms. Another embodiment of a feeding apparatus comprises a feeding tube. The feeding tube includes one or more surfaces having a constituent polymer matrix. The constituent polymer matrix includes one or more anti-biofim mechanisms. One embodiment of a method of preparing an extended-longevity feeding tube comprises the step of adding one or more anti-biofilm mechanisms to one or more surfaces of a feeding tube. One embodiment of a method of placing an extended-longevity feeding tube, comprises steps. The steps include creating an opening in a patient and inserting a feeding tube in the patient. The feeding tube includes one or more surfaces having one or more anti-biofilm mechanisms. One embodiment of a method of using an extended-longevity feeding tube comprises components. These components include installing a feeding tube in a patient. The feeding tube includes one or more surfaces having one or more anti-biofilm mechanisms. Another component is feeding the patient with the tube.

Feeding tubes as described in the aforementioned embodiments are a new approach which offer several important advantages over existing technology.

### BRIEF DESCRIPTION OF THE DRAWINGS

FIG. 1 shows a radial cross section of feeding tube with an anti-biofilm mechanism as part of constituent polymer.

FIG. 2 shows a radial cross section of feeding tube (cut distal to the bumper).

FIG. 3 shows a radial cross section of feeding tube (view proximal to the bumper).

FIG. 4 shows a feeding tube in longitudinal cross section.

FIG. 5 shows another embodiment of FIG. 2.

FIG. 6 shows another embodiment of FIG. 4.

FIG. 7 shows a radial cross section of feeding tube (cut distal to the bumper) with reservoirs.

FIG. 8 shows another embodiment of FIG. 7.

FIG. 9 shows a skin level device (also known as a “button”).

FIG. 10 shows a skin level device with a balloon.

FIG. 11 shows placement of extended-longevity gastrostomy tube.

FIG. 12 shows extended-longevity gastrostomy tube in place in the stomach.

FIG. 13 shows use of extended-longevity gastrostomy tube.

FIG. 14 shows use of extended-longevity skin level device.

FIG. 15 is a flow chart describing one embodiment of a process for practicing the current invention.

### DETAILED DESCRIPTION

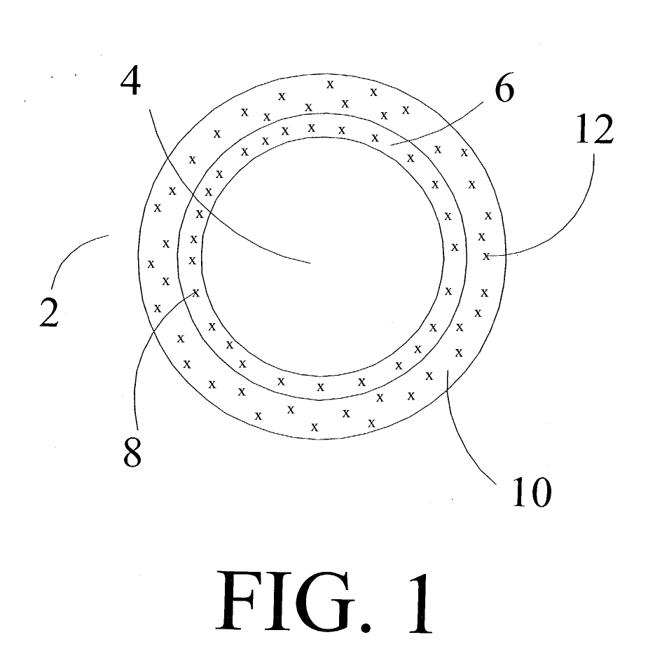

FIG. 1 is a radial cross-section of a feeding tube 2. In the center of the tube is the lumen 4. Surrounding the lumen 4 is an internal surface treated layer 6. Therapeutic agent 8 is within the layer of surface treatment 6. Surrounding the internal surface treated layer 6 is the constituent polymer layer 10. Bulk therapeutic agent is within the constituent polymer layer 12.

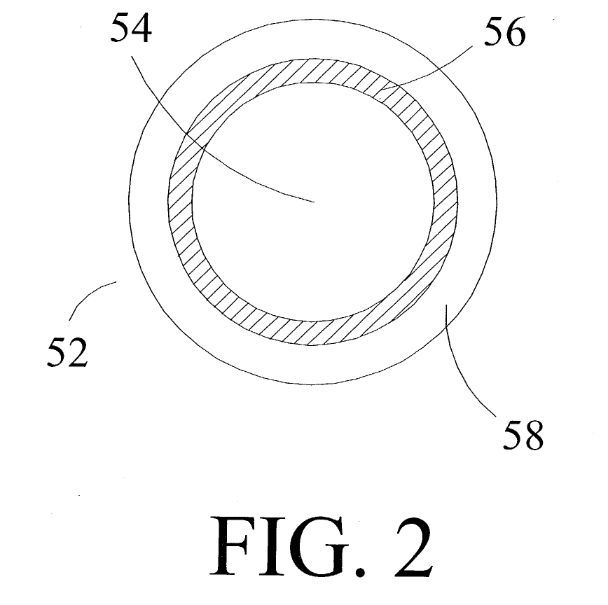

FIG. 2 is a distal radial cross-sectional view of a feeding tube 52. In the center of the tube is the lumen 54. Surrounding the lumen 54 is an internal surface treated layer 56. Surrounding the internal surface treated layer 56 is the tube body 58.

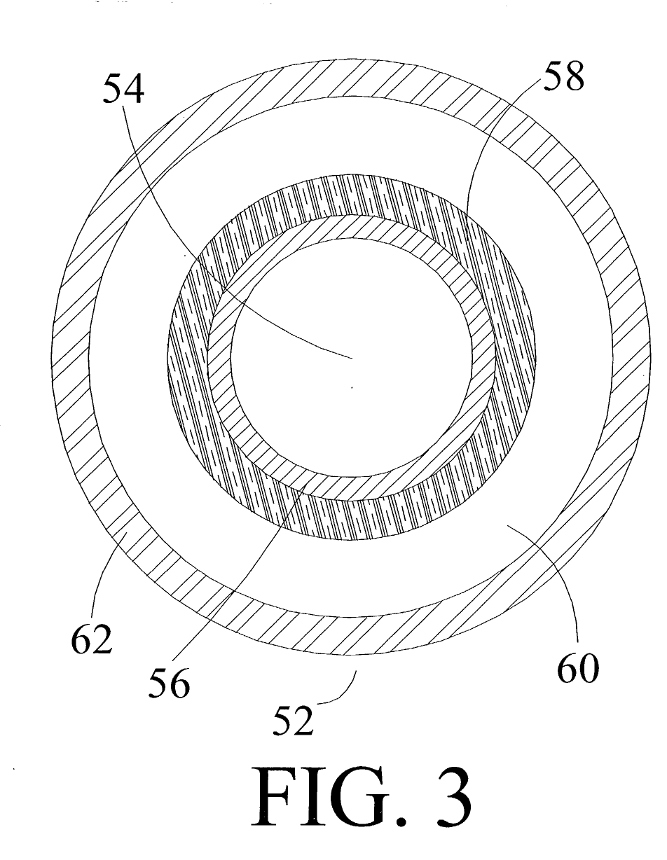

FIG. 3 is a proximal radial cross-sectional view of a feeding tube 52. In the center of the tube is the lumen 54. Surrounding the lumen 54 is an internal surface treated layer 56. Surrounding the internal surface treated layer 56 is the tube body 58. Surrounding the tube body 58 is the surface treated internal bumper surface 60. Meeting the surface treated internal bumper surface is the external surface treated bumper surface 62.

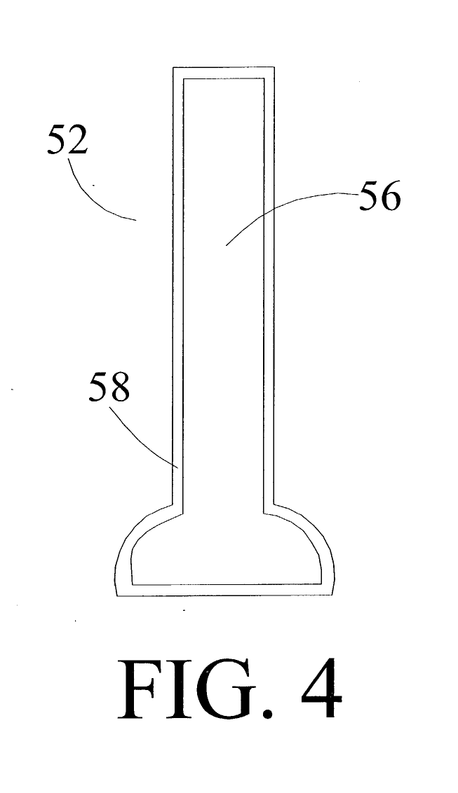

FIG. 4 is a longitudinal cross-sectional view of a feeding tube 52. In the center of the tube is the internal surface treated layer 56. Surrounding the internal surface treated layer 56 is the tube body 58.

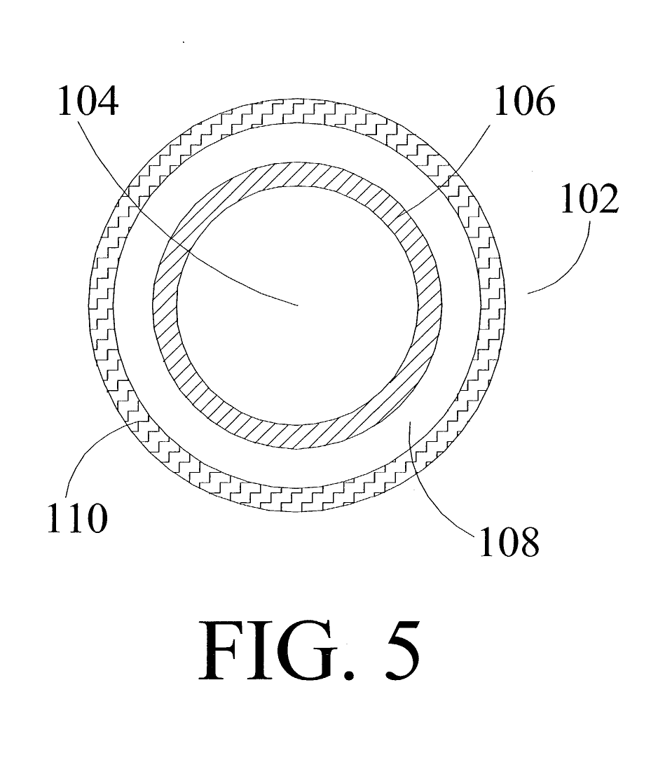

FIG. 5 shows an alternative embodiment of the present invention. It shows a distal radial cross-sectional view of a feeding tube 102. In the center of the tube is the lumen 104. Surrounding the lumen 104 is the internal surface treated layer 106. Surrounding the internal surface treated layer 106 is the tube body 108. Surrounding the tube body 108 is the external surface treated layer 110.

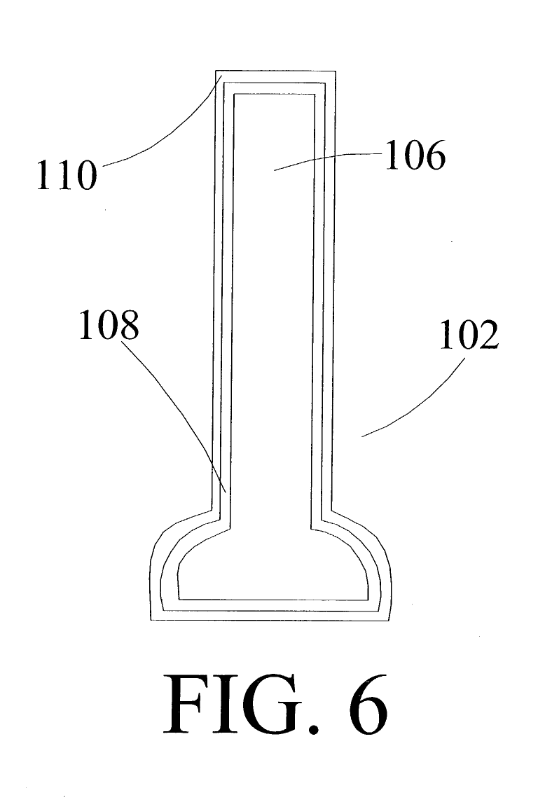

FIG. 6 shows a longitudinal cross-sectional view of the feeding tube 102 in FIG. 5. In the center of the tube is the internal surface treated layer 106. Surrounding the internal surface treated layer 106 is the tube body 108. Surrounding the tube body 108 is the external surface treated layer 110.

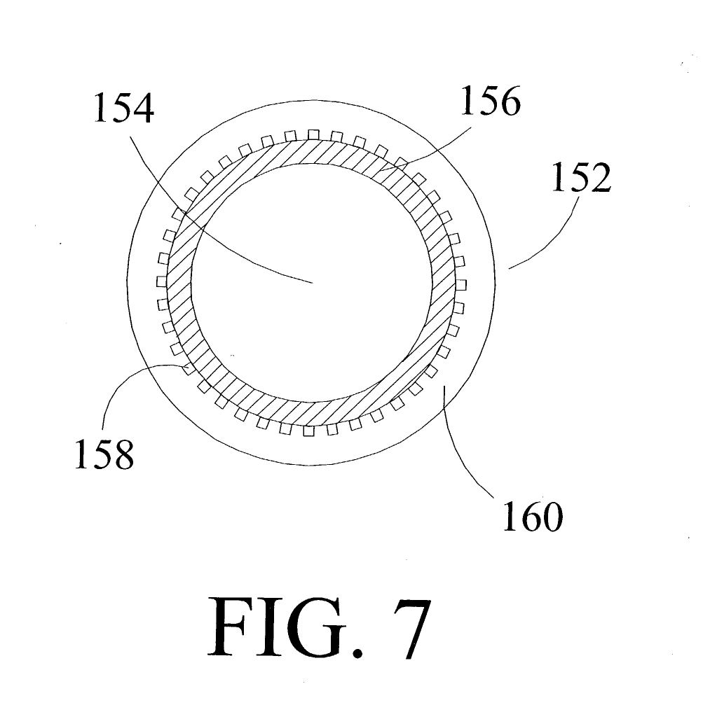

FIG. 7 is an alternative embodiment of the present invention. It shows a distal cross-sectional view of a feeding tube 152. In the center of the tube is the lumen 154. Surrounding the lumen 154 is the internal surface treated layer 156. Surrounding the internal surface treated layer 156 are reservoirs 158. Surrounding each reservoir on three sides is the tube body 160.

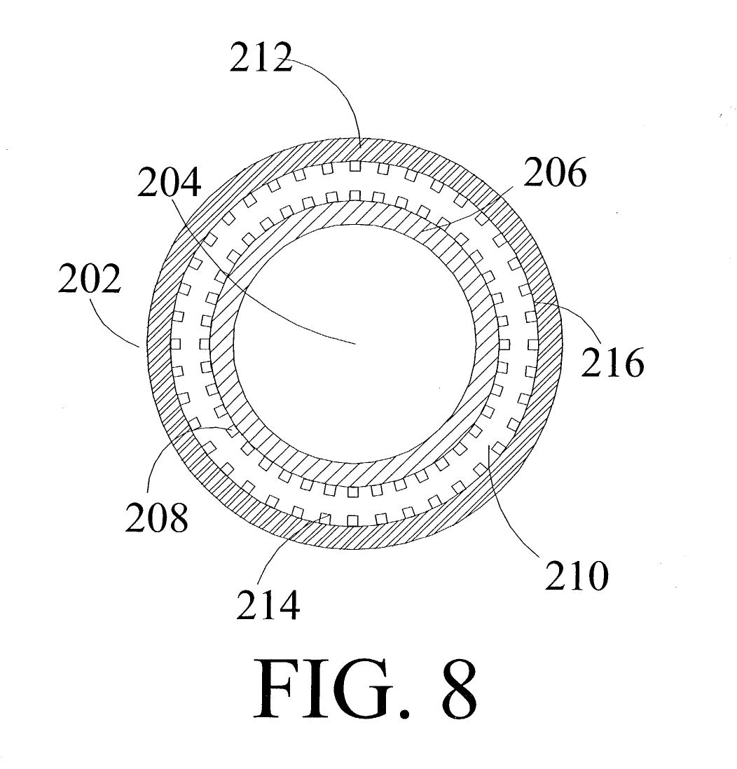

FIG. 8 is an alternative embodiment of the present invention. It shows a distal cross-sectional view of a feeding tube 202. In the center of the tube is the lumen 204. Surrounding the lumen 204 is the internal surface treated layer 206. Surrounding the internal surface treated layer 206 are internal reservoirs 208. Surrounding each internal reservoir 208 on three sides is the tube body 210. Surrounding the tube body 210 is the external surface treated layer 212. External reservoirs 214 appear adjacent to the inner border 216 of the external surface treated layer 212.

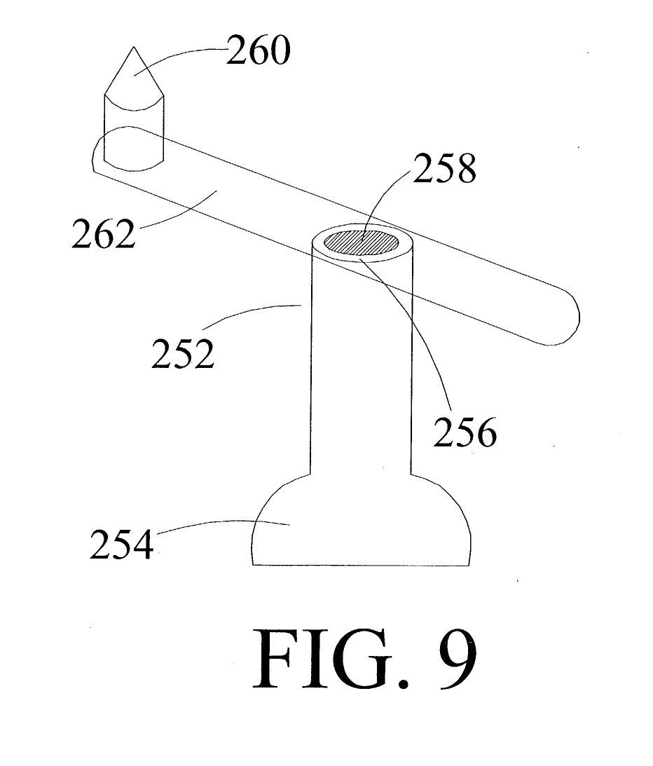

FIG. 9 is a skin level feeding tube 252. It shows the external surface treated skin level feeding tube bumper surface 254. Distal to the external surface treated bumper surface 254 is the skin level feeding tube body 256. At the far distal end of the external skin level feeding tube 252 is the lumen and the internal surface treated layer 258. The button plug 260 is attached to the button flap 262 and folds over to close the lumen 258 in between feedings following placement in the patient.

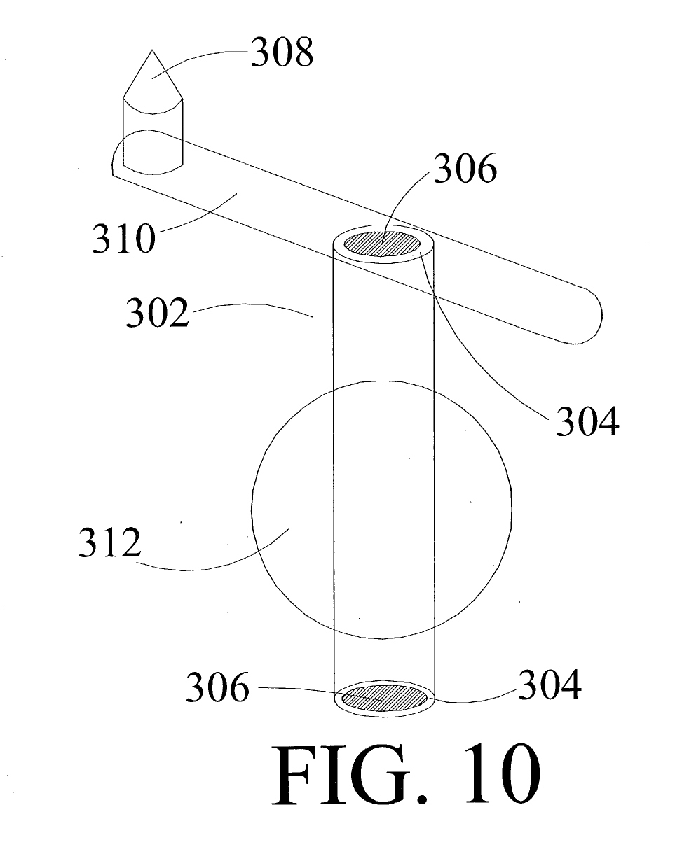

FIG. 10 is another embodiment of a skin level feeding tube 302. At opposite ends of the skin level feeding tube body 304 is the lumen and the internal surface treated layer 306. The button plug 308 is attached to the button flap 310 and folds over to close the lumen 306 in between feedings following placement in the patient. An inflatable balloon 312 surrounds the skin level feeding tube body 304.

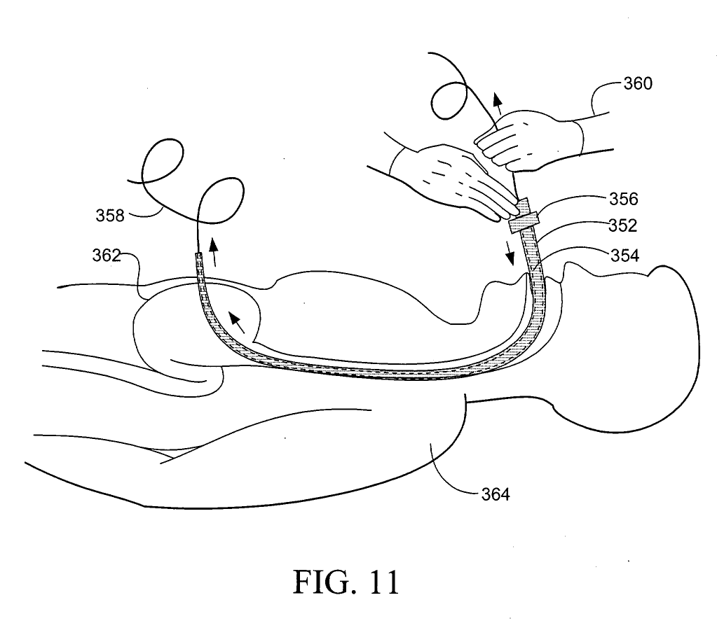

FIG. 11 is a diagram of a feeding tube 352 with an internal surface treated layer 354 being placed in the patient via percutaneous endoscopic gastrostomy (PEG). The bumper 356 is also surface treated. Using a guidewire 358, the operator 360 places the feeding tube 352 into the stomach 362 of the patient 364.

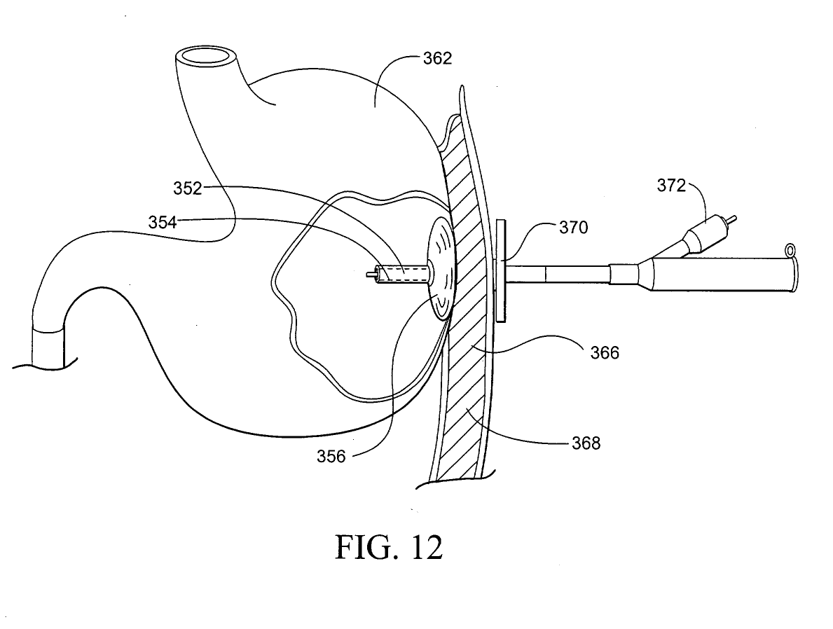

FIG. 12 is a diagram of feeding tube 352 with internal surface treated layer 354 in place in the stomach 362. The feeding tube 352 passes through the stoma 366 in the abdominal wall 368. The bumper 356 is also surface treated. A crossbar 370 holds the feeding tube 352 in place. An adaptor 372 is attached to the distal end of the feeding tube 352.

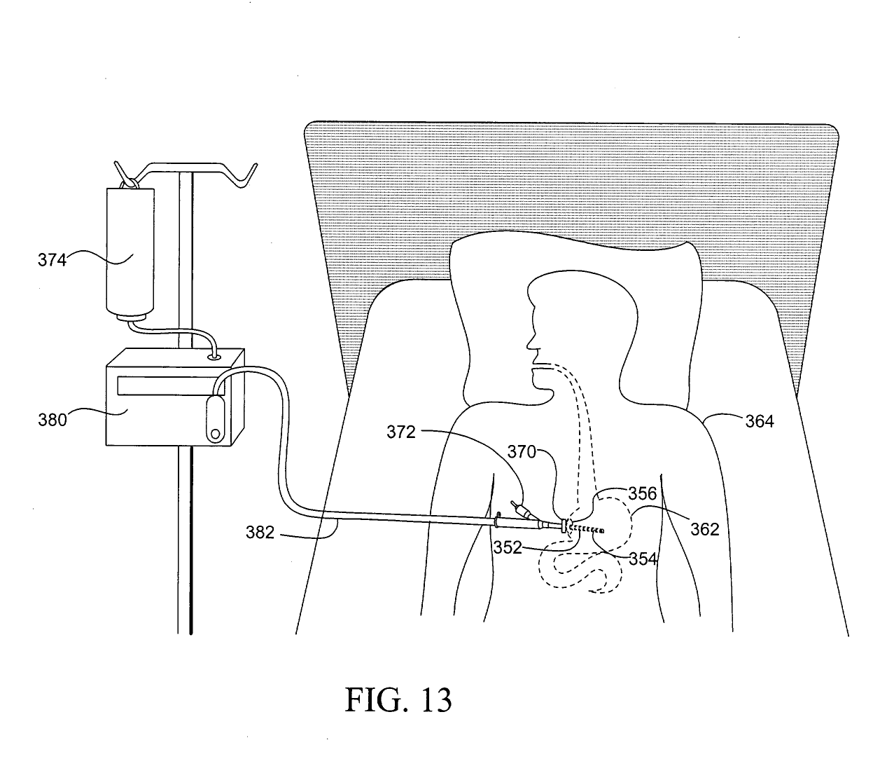

FIG. 13 is a diagram of a patient 364 receiving feedings via a feeding tube 352 with internal surface treated layer 354. Enteral feedings pass from the enteral feeding container 374 via an enteral feeding pump 380 via an uncoated feeding tube 382 through the feeding tube 352 with internal surface treated layer into the stomach 362 of the patient 364. The bumper 356 is also surface treated. A crossbar 370 holds the feeding tube 352 in place. An adaptor 372 is attached to the distal end of the feeding tube 352.

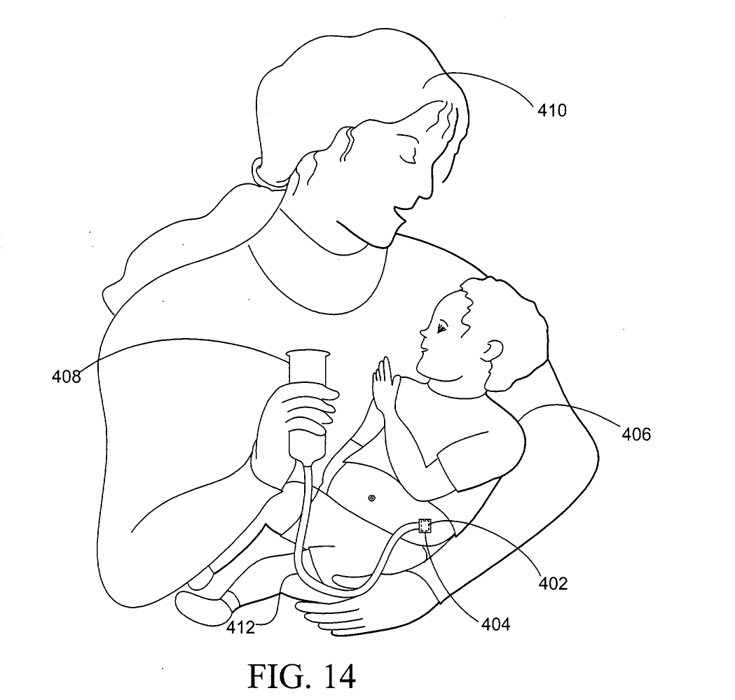

FIG. 14 is a diagram of a surface treated layer skin-level device 402 entering the stoma 404 of the patient 406. Enteral feeding container 408 is held by the caregiver 410. The surface treated skin-level device 402 is connected to the enteral feeding container by an uncoated feeding tube 412.

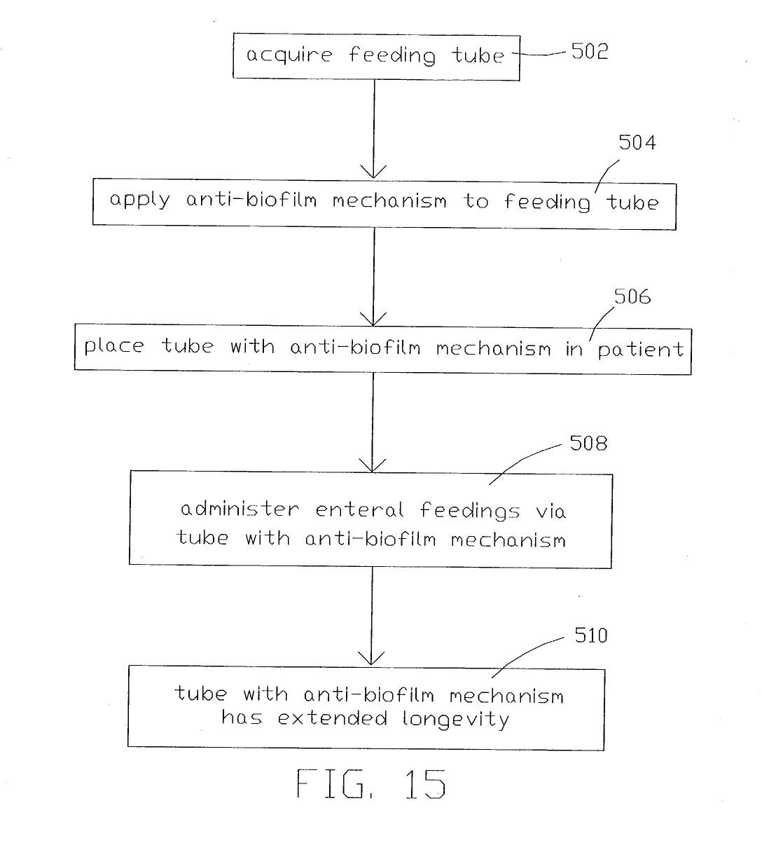

FIG. 15 is a flow chart which explains the operation of the present invention. In step 502, the feeding tube is acquired. In step 504, the anti-biofilm mechanism is applied. In step 506, the tube with anti-biofilm mechanism is placed in the patient. In step 508, enteral feedings are administered via tube with anti-biofilm mechanism. In step 510 the tube has achieved extended longevity. Subsequent to step 510, the process may be repeated.

In this application, it may be desired to deliver a therapeutic agent to the internal and/or external surface of a feeding tube. This delivery can occur at any time prior to or after placement into the patient.

The conventional approach of feeding tube design leaves the tube vulnerable to fungal colonization and subsequent rapid deterioration of the structural and functional integrity. The ideal surface treatement should preferably be able to alter the properties of the tube in such a manner as to allow strong adherence of a therapeutic agent or as to prevent or delay the formation and proliferation of biofilm on the tube surface. If a therapeutic agent is applied to the tube via a surface treatment, then it should preferably be capable of retaining the drug at a sufficient load level to obtain the required dose, be able to release the drug in a controlled way over a period of several weeks, and be thin in order to minimize the increase in profile. In addition the surface treatment and/or therapeutic agents should preferably not contribute to any adverse response by the body (i.e. should be non-thrombogenic, non-inflammatory, etc.).

Numerous agents can be inhibitors of antimicrobial biofilm formation, including caspofungin, a glucan synthesis inhibitor of the echinocandin structural class. Echinocandins are presumed to block fungal cell wall synthesis by inhibiting the enzyme 1,3-beta glucan synthase. This novel mechanism permits echinocandins to be effective against most commonly encountered fungi that have become resistant to currently used antifungal drugs. Caspofungin is active against Candida spp., including species that are resistant (Candida krusei), or isolates that are less susceptible (Candida dubliniensis, Candida glabrata) to azoles, or resistant to amphotericin B) (Nelson P W, Lozano-Chiu M, Rex J H. In vitro growth-inhibitory activity of pneumocandins L-733,560 and L-743,872 against putatively amphotericin B- and fluconazole-resistant Candida isolates: influence of assay conditions. Journal of Medical and Veterinary Mycology 1997;35:285-7; Bachmann S P, Perea S, Kirkpatrick W R, Patterson T F, Lopez-Ribot J L. In vitro activity of cancidas (MK-0991) against Candida albicans clinical isolates displaying different mechanisms of azole resistance. In Program and Abstracts of the Fortieth Interscience Conference on Antimicrobial Agents and Chemotherapy, Toronto, Ontario, Canada, 2000. Abstract J-196, p.352. American Society for Microbiology, Washington, D.C., USA. Espinel-Ingroff A. Comparison of in vitro activities of the new triazole SCH56592 and the echinocandins MK-0991 (L-743,872) and LY303366 against opportunistic filamentous and dimorphic fungi and yeasts. Journal of Clinical Microbiology 1998;36:2950-6; Sutton D A, Rinaldi M G, Fothergill A W. In vitro activity of the echinocandin caspofungin (MK-0991) against refractory clinical isolates of Candida and Aspergillus species. In Program and Abstracts of the Forty-first Interscience Conference on Antimicrobial Agents and Chemotherapy, Chicago, Ill., USA, 2001. Abstract J-113, p. 361. American Society for Microbiology, Washington, D.C., USA; Vazquez J A, Lynch M, Boikov D, Sobel J D. In vitro activity of a new pnuemocandin antifungal, L-743,872, against azole-susceptible and -resistant Candida species. Antimicrobial Agents and Chemotherapy 1997;41:1612-4). Caspofungin is manufactured by Merck Research Laboratories.

Two other members of the echinocandin family include micafungin and anidulafungin. Micafungin is manufactured by Fujisawa. Anidulafungin is manufactured by Eli Lilly Pharmaceuticals.

Another new promising drug with respect to inhibition of fungal biofilms is liposomal amphotericin B, a unilamellar (single-layer) liposomal formulation of amphotericin B. Liposomal amphotericin B is manufactured by Gilead Sciences.

Using time-kill studies, caspofungin was compared to fluconazole and amphotericin with respect to in vitro activity against Candida albicans biofilms (Ramage G, VandeWalle K, Bachmann S P, Wickes B L, Lopez-Ribot J L. In vitro pharmacodynamic properties of three antifungal agents against preformed Candida albicans biofilms determined by time-kill studies. Antimicrob Agents Chemother 2002 November;46(11):3634-6). Caspofungin demonstrated the most effective pharmacokinetic properties, with ≧99% killing at physiological concentrations.

Another study looked at the in vitro activity of caspofungin against Candida albicans biofilms (Bachmann S P, VandeWalle K, Ramage G, Patterson T F, Wickes B L, Graybill J R, Lopez-Ribot J L. In vitro activity of caspofungin against Candida albicans biofilms. Antimicrob Agents Chemother 2002 November;46(11):3591-6). Conventional antifungals have proven ineffective against Candida albicans biofilms. Caspofungin demonstrated potent in vitro activity against sessile Candida albicans cells within biofilms. A minimum inhibitory concentration at which 50% of the sessile cells were inhibited was well within the drug's therapeutic range. The effects of caspofungin on preformed Candida albicans biofilms were studied using scanning electron microscopy and confocal scanning laser microscopy. Caspofungin altered the cellular morphology and the metabolic status of the cells within the biofilms. Additionally, the coating of biomaterials with caspofungin had an inhibitory effect on subsequent biofilm development by Candida albicans. These findings show that caspofungin displays potent activity against in vitro Candida albicans biofilms.

Another study looked at the antifungal susceptibilities of Candida albicans and Candida parapsilosis biofilms (Kuhn D M, George T, Chandra J, Mukherjee P K, Ghannoum M A. Antifungal susceptibility of Candida biofilms: unique efficacy of amphotericin B lipid formulations and echinocandins. Antimicrob Agents Chemother 2002 June;46(6):1773-80). Caspofungin and micofungin (echinocandins) showed activity against Candida biofilms. Lipid formulations of amphotericin B (liposomal amphotericin B and amphotericin B lipid complex) also showed activity against Candida biofilms. Confocal scanning laser microscopy demonstrated the drug effects on cell structure. All Candida biofilms were resistant to fluconazole, nystatin, chlorhexidine, terbenafine, amphotericin B, voriconazole, and ravuconazole.

The observations and results obtained in these three recent in vitro studies clearly support the potential use of caspofungin and/or lipid formulations of amphotericin B (liposomal amphotericin B and amphotericin B lipid complex) as therapeutic agents in the extension of feeding tube longevity.

Local delivery of therapeutic agents such as caspofungin can occur from a surface treatment applied to the internal and/or external surface of a feeding tube, button, and/or bumper. This can include co-mixture with polymers (both degradable and nondegrading) to hold the drug to the feeding tube or entrapping the drug into the feeding tube body which has been modified to contain micropores or reservoirs, as will be explained further herein. Other possible techniques include the covalent binding of the drug to the feeding tube via solution chemistry techniques (such as via the Carmeda process) or dry chemistry techniques (e.g. vapour deposition methods such as rf-plasma polymerization) and combinations thereof.

Placement of an extended-longevity feeding tube can occur endoscopically, surgically, radiologically, and via a transnasal approach. Since an anti-biofilm mechanism has been applied to the feeding tube prior to placement, the tube will not be vulnerable to biofilm colonization at the time of placement.

An extended-longevity feeding tube can be used to administer bolus, continuous, or gravity feedings. All aspects of tube use including pre-feeding checking, maintenance, and post-feeding checking can be done with an extended-longevity feeding tube.

Delivery of One or more Anti-biofilm Mechanisms from Constituent Polymer Matrix with or without an Overlying Surface-treated Layer

An anti-biofilm mechanism, with or without an overlying surface-treated layer containing an anti-biofilm mechanism, can be treated by delivery from a feeding tube polymer matrix. Such a delivery technique is described in Wright et al U.S. Pat. No. 6,273,913, incorporated herein by reference in its entirety. Solution of anti-biofilm mechanism, prepared in a solvent miscible with polymer carrier solution, is mixed with solution of polymer at final concentration range 0.001 weight % to 30 weight percentage of anti-biofilm mechanism or in an amount deemed sufficient to one skilled in the art.

Polymers are biocompatible (i.e., not elicit any negative tissue reaction) and degradable, such as lactone-based polyesters or copolyesters, e.g., polylactide, polycaprolactonglycolide, polyorthoesters, polyanhydrides; polyaminoacids; polysaccharides; polyphosphazenes; poly (ether-ester) copolymers, e.g., PEO-PLLA, or blends thereof. Nonabsorbable biocompatible polymers are also suitable candidates. Other polymers include polydimethylsiolxane; poly(ethylene-vingylacetate); acrylate based polymers or copolymers, e.g., poly(hydroxyethyl methylmethacrylate, polyvinyl pyrrolidinone; fluorinated polymers such as polytetrafluoroethylene; cellulose esters. The polymer/anti-biofilm mechanism mixture is applied to the surfaces of the feeding tube by either dip-coating, or spray coating, or brush coating or dip/spin coating or combinations thereof, and the solvent allowed to evaporate to leave a film with entrapped anti-biofilm mechanism.

It is useful to have the anti-biofilm mechanism applied with enough specificity and enough concentration to provide an effective dosage to inhibit or delay bacterial and/or fungal biofilm colonization.

Another method of preparing an anti-biofilm mechanism, with or without overlying surface treatment, as part of a feeding tube constituent matrix utilizes a method described in Schierholz et al (Schierholz J M, Steinhauser H, Rump A F E, Berkels R, Pulverer G. Controlled release of antibiotics from biomedical polyurethanes: morphological and structural features. Biomaterials 1997;18(12):839-844). Polyurethane ‘Walopur’ (Fa. Wolff, Walsrode, Germany) is an elastomeric biomaterial, consisting of aromatic polyethers (poly(oxytetramethylene glycol)) and a basic compound (diisocyanodiphenylmethane). It is freely soluble in dimethylformamide (DMF). An anti-biofilm mechanism can be selected for incorporation into the medical polyurethane. An example of an anti-biofilm mechanism is the antifungal, caspofungin. Contaminants in polyurethane can be extracted for twenty-four hours in a water/EtOH (1:1, reflux, 82° C.) or in a mixture deemed suitable to one skilled in the art. The purified polyurethane is then dissolved in DMF (reflux, 102° C.) or in a solution deemed suitable to one skilled in the art. Various amounts of anti-biofilm mechanism can be added to the solution (2, 4, 5, 7.5 and 10% w/w drug/polymer or an amount deemed suitable to one skilled in the art), dissolved or suspended under stirring. DMF is evaporated at 50° and 400 mbar for twenty-four hours below a level of 4 ppm (measured by high-performance liquid chromatography (HPLC) or under other conditions deemed suitable to one skilled in the art). The anti-biofilm mechanism is unaffected by DMF or elevated temperature.

It should be evident to those skilled in the art that methods of delivering one or more anti-biofilm mechanisms from a constituent polymer matrix with or without an overlying surface-treated layer vary considerably. Therefore, the present invention is not limited to these two particular variations of delivering one or more anti-biofilm mechanisms from a constituent polymer matrix with or without an overlying surface-treated layer.

Delivery of One or more Anti-biofilm Mechanisms via One or more Reservoirs

In another embodiment of the invention, one or more anti-biofilm mechanisms can be delivered from reservoirs in a feeding tube with or without an overlying surface treatment. Such a delivery technique is described in Wright et al U.S. Pat. No. 6,273,913. Feeding tube, whose body has been modified to contain micropores or reservoirs dipped into a solution of an anti-biofilm mechanism such as caspofungin, range 0.001 wt % to saturated or in an amount sufficient to someone skilled in the art, in organic solvent such as acetone or methylene chloride, for sufficient time to allow solution to permeate into the pores. (The dipping solution can also be compressed to improve the loading efficacy.) After solvent has been allowed to evaporate, the feeding tube is dipped briefly in fresh solvent to remove excess surface bound anti-biofilm mechanism. A solution of polymer is applied to the feeding tube as detailed above. Polymers are biocompatible (i.e., not elicit any negative tissue reaction) and degradable, such as lactone-based polyesters or copolyesters, e.g., polylactide, polycaprolactonglycolide, polyorthoesters, polyanhydrides; polyaminoacids; polysaccharides; polyphosphazenes; poly (ether-ester) copolymers, e.g., PEO-PLLA, or blends thereof. Nonabsorbable biocompatible polymers are also suitable candidates. Other polymers include polydimethylsiolxane; poly(ethylene-vingylacetate); acrylate based polymers or copolymers, e.g., poly(hydroxyethyl methylmethacrylate, polyvinyl pyrrolidinone; fluorinated polymers such as polytetrafluoroethylene; cellulose esters. This outerlayer of polymer will act as diffusion-controller for release of anti-biofilm mechanism.

It is useful to have the anti-biofilm mechanism applied with enough specificity and enough concentration to provide an effective dosage to inhibit or delay bacterial and/or fungal biofilm colonization. In this regard, the reservoir size in the tube should be kept at a size of about 0.0005″ to about 0.003″ or at a size deemed suitable to one skilled in the art. Then, it should be possible to adequately apply the anti-biofilm mechanism dosage at the desired location and in the desired amount.

As seen in FIG. 7 a feeding tube body 160 can be modified to have one or more reservoirs 158. Each of these reservoirs can be open or closed as desired. These reservoirs can hold one or more anti-biofilm mechanisms to be delivered.

It should be evident to those skilled in the art that methods of delivering one or more anti-biofilm mechanisms via one or more reservoirs vary considerably. Therefore, the present invention is not limited to this one particular variation of delivering one or more anti-biofilm mechanisms via one or more reservoirs.

Methods of Adding One or more Anti-biofilm Mechanisms to One or more Surfaces

### EXAMPLE 1

Adding One or more Anti-biofilm Mechanisms via Formation of a Covalent Drug Tether from which One or more Anti-biofilm Mechanisms can be Lysed

An anti-biofilm mechanism of a feeding tube can also be achieved by formation of a covalent drug tether from which one or more anti-biofilm mechanisms can be lysed. Such a delivery technique is described in Wright et al U.S. Pat. No. 6,273,913. One example of an anti-biofilm mechanism that can be used is caspofungin. Caspofungin, in a quantity deemed sufficient to one skilled in the art, is modified to contain a hydrolytically or enzymatically labile covalent bond for attaching to the surface of the feeding tube which itself has been chemically derivatized to to allow covalent immobilization. Covalent bonds such as ester, amides or anhydrides may be suitable for this.

It is useful to have the anti-biofilm mechanism applied with enough specificity and enough concentration to provide an effective dosage to inhibit or delay bacterial and/or fungal biofilm colonization.

It should be evident to those skilled in the art that methods of forming a covalent drug tether from which one or more anti-biofilm mechanisms can be lysed vary considerably. Therefore, the present invention is not limited to this one particular variation of forming a covalent drug tether from which one or more anti-biofilm mechanisms can be lysed.

### EXAMPLE 2

Adding One or more Anti-biofilm Mechanisms via Intraluminal Delivery from a Polymeric Sheet

An anti-biofilm mechanism of a feeding tube can also be achieved by applying a polymeric sheet containing a therapeutic agent. Such a delivery technique is described in Wright et al U.S. Pat. No. 6,273,913. Formation of a polymeric sheet with an anti-biofilm mechanism such as caspofungin is combined at concentration range 0.001 weight % to 30 weight % of drug or in an amount deemed suitable to one skilled in the art, with a degradable polymer such as poly(caprolactone-glycolide) or non-degradable polymer, e.g., polydimethylsiloxane, and mixture cast as a thin sheet, thickness range 10μ to 1000 μ or in a thickness range deemed suitable to one skilled in the art. The resulting sheet can be wrapped intraluminally on the feeding tube. Preference would be for the absorbable polymer.

It is useful to have the anti-biofilm mechanism applied with enough specificity and enough concentration to provide an effective dosage to inhibit or delay bacterial and/or fungal biofilm colonization.

It should be evident to those skilled in the art that methods of intraluminal delivery from a polymeric sheet vary considerably. Therefore, the present invention is not limited to this one particular variation of intraluminal delivery.

### EXAMPLE 3

Adding One or more Anti-biofilm Mechanisms via a Bonding Process

An anti-biofilm mechanism of a feeding tube can also be achieved by a bonding process. In a broad sense, chemical bonds can be ionic or covalent. An example of a bonding process that can be used to treat the surface of a feeding tube is described in Greco et al U.S. Pat. No. 4,740,382, which is incorporated herein by reference in its entirety. Feeding tubes are placed in a solution of cationic surfactant, such as a 5% ethanol solution of tridodecylmethyl ammonium chloride (TDMAC) for a period of time of from 5 to 120 minutes, preferably about 30 minutes, or for a duration of time deemed suitable to one skilled in the art, and at a temperature of from 0° to 55° C., preferably at ambient temperature, or at a temperature deemed suitable to one skilled in the art. The feeding tubes are air dried and thoroughly washed in distilled water to remove excess TDMAC.

The feeding tubes having an absorbed coating of TDMAC are then placed in a solution of anti-biofilm mechanism such as negatively-charged caspofungin, in an amount deemed suitable by one skilled in the art, for a period of time from 5 to 120 minutes, preferably 60 minutes, or for a duration of time deemed suitable to one skilled in the art, at a temperature from 0° to 35° C., preferably 25° C., or at a temperature deemed suitable to one skilled in the art. The thus treated tubes are then thoroughly washed, preferably in distilled water to remove unbound anti-biofilm mechanism, it being understood that not all of the unbound anti-biofilm mechanism material is removed from the thus treated tubes.

The feeding tubes having TDMAC/anti-biofilm mechanism compound bounded thereto are immersed in a slurry of a particulate insoluble cationic exchange compound, such as Sepharose-CM, cross-linked agarose having carboxyl methyl groups (CH₂——COO—) attached thereto for a period of time from 6 to 72 hours, preferably 20 hours, or for a duration of time deemed suitable by one skilled in the art, at a temperature of from 0° to 35° C., preferably 25° C., or at a temperature deemed suitable by one skilled in the art. The cationic exhange compound is in the form of beads having a particle size distribution of from 5 to 40 microns, or having a particle size distribution deemed suitable by one skilled in the art, and is commercially available in such particle size distribution. The thus treated tubes are then thoroughly washed in distilled water.

It should be evident to those skilled in the art that bonding processes vary considerably. Therefore, the present invention is not limited to this one particular variation of a bonding process.

### EXAMPLE 4

Adding One or more Anti-biofilm Mechanisms via a Vacuum Deposition or a Vacuum Coating Process

An anti-biofilm mechanism of a feeding tube can also be achieved by vacuum deposition. Surface modification of a feeding tube via vacuum deposition deposits a thin coating of metal onto the surface of the tube by condensation on a cool work surface in vacuum. One example of vacuum deposition is anodic vacuum arc deposition. Such a vacuum coating technique is described in Arweiler-Harbeck et al (Arweiler-Harbeck D, Sanders A, Held M, Jerman M, Ehrich H, Jahnke K. Does metal coating improve the durability of silicone voice prostheses? Acta Otolaryngol 2001 July;121(5):643-6). Feeding tube is placed above the anode in a vacuum chamber. The anode is heated by means of particle bombardment from the cathode and pure titanium is evaporated and ionized. The ionized anodic titanium expands into the ambient vacuum forming an anodic arc, which deposits onto the silicone surface. The feeding tube should be placed or rotated in such a manner that a homogenous coating of the tube in the desired regions is achieved. Other metals can also be used. Examples of such metals include gold and aluminum. In addition to these coatings, various process parameters should be employed. Coating is differentiated from pretreatment. With regard to coating itself, current (40-100A), plasma power (40W) and coating thickness should be measured. Other currents or plasma power can be utilized in an amount deemed sufficient to one skilled in the art. With regard to pretreatment, air pressure (also possible without Pa), plasma power (W) and DC bias (V) are of major importance. These parameters should be adjusted to amounts deemed necessary by one skilled in the art.

It should be evident to those skilled in the art that vacuum deposition methods vary considerably. Therefore, the present invention is not limited to this one particular variation of vacuum deposition.

### EXAMPLE 5

Adding One or more Anti-biofilm Mechanisms via Hydrogel Encapsulation

An anti-biofilm mechanism feeding tube can also be achieved by a hydrogel encapsulation method. Such a hydrogel encapsulation method is described in DiCosmo et al U.S. Pat. No. 6,475,516, incorporated herein by reference in its entirety.

All steps prior to preparation of feeding tube are done as described by U.S. Pat. No. 6,475,516.

Preparation of Feeding Tube

Feeding tube material that is to be coated with PEG-gelatin gel is first spin-coated with 10.mu.L of AFB-gelatin (5 mg/mL;α=55%) and dried under vacuum for 1 hour. All coated sections are exposed to UV light (254 nm) for 3 minutes and rinsed with water. Subsequently, feeding tube pieces are spin-coated with 60 μL of fluid PEG-Gelatin or PEG-gelatin-liposome mixture and incubated at 4° C. for 15 minutes. Incubation may occur at temperatures from 4-10° C. Gels are polymerized by submersing feeding tube sections in 200 mM Borate buffer (pH 8.5) for 1 hr. Residual p-nitrophenol is leached from the gels by incubation at room temperature in 10% sucrose (pH 4.0) for 12 hrs, with four changes of medium. The absence of p-nitrophenol is confirmed by negligible absorbance of the dialysate at 410 nm. Liposomes in suspension and those entrapped within PEG-gelatin gels are loaded with an anti-biofilm mechanism such as caspofungin according to the remote-loading technique described in Y. K. Oh, D. E. Nix, and R. M. Straubinger, “Formulation and efficacy of liposome-encapsulated antibiotics for therapy of intracellular Mycobacterium avium infection,” Antimicrob Agents Chemother, 39:2104-2111 (1995). Feeding tube pieces are placed in 10% sucrose solution (pH 7.5) containing 2 mM caspofungin, while for liposomes in suspension, an appropriate amount of drug is added to make the suspension 2 mM in caspofungin. Incubation in both cases proceeds for 1 hour at 45° C. The liposome suspension is centrifuged at 3000*g for 5 minutes to pellet drug crystals and the supernatant is then applied to a G-50 column (1*10 cm) to remove unentrapped caspofungin.

Dehydrated hydrogels are prepared by drying coated feeding tube sections in an oven at 35° C. for 2.5 hours. The dried gels are then rehydrated in Tris buffer (10 mM Tris, 110 mM NaCl, pH 7.4) or in concentrated caspofungin-HCl solution (25 mg/mL) as required. The temperature during the rehydration process is maintained at 45° C.

The quantity of anti-biofilm mechanism loaded on the substrate can be increased or decreased. Greater concentrations of anti-biofilm mechanism can be loaded by increasing the amount of anti-biofilm mechanism encapsulated and mixed into the hydrogel. For example, concentrations up to about 1,000 μg (1.0 mg) per cm² or more of an anti-biofilm mechanism can be loaded on substrates with the methods of the present invention; and that concentrations of up to about 10,000 μg/cm³ or more can be loaded on substrates. A preferred concentration range of anti-biofilm mechanism loaded on such substrates is about 10-1,000 μg/cm².

Similarly, quantities of therapeutic agent can be increased by increasing the quantity of gel immobilized on the surface of the substrate. Generally, hydrogel layers of about 0.5-10 mm thick can be loaded on substrates to effect the desired drug delivery and therapeutic results; preferred layers are in the range of about 1-5 mm; and especially preferred layers are about 2-4 mm.

Thus, one of skill in the art will appreciate that the present methods and devices afford highly versatile means for loading high concentrations of anti-biofilm mechanism, and of varying the concentration of anti-biofilm mechanism, on a substrate or on a specific area of a substrate.

It should be evident to those skilled in the art that hydrogel encapsulation methods vary considerably. Therefore, the present invention is not limited to this one particular variation of hydrogel encapsulation.

### EXAMPLE 6

Adding One or more Anti-biofilm Mechanisms via Solvent Casting

A feeding tube anti-biofilm mechanism can also be achieved by a solvent casting method. Such a solvent casting method is described in Gollwitzer H et al (Gollwitzer H, Ibrahim K, Meyer H, Mittelmeier W, Busch R, Stemberger A. Antibacterial poly (d,l-lactic acid) coating of medical implants using a biodegradable drug delivery technology. Journal of Antimicrobial Chemotherapy 2003;51:585-591. The Resomer R203 is a polymer of PDLLA with a molecular weight of 29,000 Da. It is commercially available and can be purchased from Boehringer Ingelheim (Ingelheim, Germany). A racemic mixture of the D- and L- enantiomers of lactic acid comprises the polymer and serves as a biodegradable coating for feeding tubes. A solvent casting technique is used to coat feeding tubes with PDLLA. The drug-carrier is dissolved in ethyl-acetate (Sigma-Aldrich AG, Deisenhofen, Germany) at a concentration of 133.3 mg/mL. To prevent evaporation of the organic solvent and a subsequent increase in the polymer concentration the coating solution is maintained on dry ice. To create a local delivery system 5% (w/w) of an anti-biofilm mechanism, such as caspofungin, is added to the polymer solution. In order to achieve a dense and regular polymer coating, the feeding tube is coated by two or more dip-coating procedures to achieve a dense and regular polymer coating. All coating steps are carried out under aseptic conditions with laminar air-flow.

It should be evident to those skilled in the art that solvent casting methods vary considerably. Therefore, the present invention is not limited to this one particular variation of solvent casting.

### EXAMPLE 7

Adding One or more Anti-biofilm Mechanisms via Dip Coating

A feeding tube anti-biofilm mechanism can also be achieved by dip coating (also known as dipping or immersion coating). This method applies a coating to a feeding tube by immersion into a tank of metallic or nonmetallic material, then chilling the adhering melt. A feeding tube is dipped at least once in to solution. Liquid dip coating equipment that can be used to prepare an extended-longevity feeding tube can range from a simple dip tank to a sophisticated electrocoating system. Since dipping is known to reduce early-onset colonization of medical devices, this simple process may be ideal for feeding tubes as they are likely colonized by biofilm during placement.

The dipping solution can contain one or more of the following anti-biofilm mechanisms: antifungal agents, antibacterial agents, metals, antiseptics, disinfectants, gene expression blockers, or therapeutic agents inhibiting the formation of granulation tissue.

Examples of typical polymers include polyurethane, ethylenevinyl acetate, silicone dispersion. Examples of antibacterials include iodine, aminoglycosides (gentamicin, tobramycin), ciprofloxacin, parabens, quaternary ammonium salts (benzalkonium chloride), chloramphenicol, and chlorhexidine. Examples of antifungals include: amphotericin B (including liposomal formulation of amphotericin B), caspofungin, anidulafungin, micafungin, nystatin, clotrimazol, ciclopiroxolamine, chlorhexedine.

Another example of dip coating a feeding tube to achieve an anti-biofilm mechanism can employ the methodology described in Raad et al published U.S. Pat. Application 2003/0078242, incorporated herein by reference in its entirety. The antiseptic compound is therefore applied on the surface of a feeding tube by simply immersing the tube in a solvent comprising an anti-biofilm mechanism such as a basic antiseptic reagent and a dye, air drying and washing out excessive antiseptic. The self-impregnating property of the dyes such as for example, the triarylmethane dyes, removes the need for another binding agent.

It should be evident to those skilled in the art that dip coating methods vary considerably. Therefore, the present invention is not limited to any one particular variation of dip coating.

### EXAMPLE 8

Adding One or more Anti-biofllm Mechanisms via Spray Coating

A feeding tube anti-biofilm mechanism can also be achieved by spray coating. For example, an anti-biofilm mechanism such as caspofungin can be sprayed onto a feeding tube. During a spray coating process, micro-sized spray particles are deposited onto the feeding tube. Air, hydraulic, or centrifugal spray coating equipment can be used to prepare an extended-longevity feeding tube. Specific examples of spray coating equipment that could be used to prepare extended-longevity feeding tubes include the following: conventional air atomize; airless; air-assisted-airless; air electrostatic; airless electrostatic; air-assisted-airless-electrostatic; high-volume low-pressure; and rotating electrostatic discs and bells.

An example of spray coating a feeding tube to achieve an anti-biofilm mechanism can employ the methodology described in Hossainy et al published U.S. Pat. Application 2001/0014717, incorporated herein by reference in its entirety.

It should be evident to those skilled in the art that spray coating methods vary considerably. Therefore, the present invention is not limited to this one particular variation of spray coating.

### EXAMPLE 9

Adding One or more Anti-biofilm Mechanisms via Laser Processes

Laser processes are another anti-biofilm mechanism that can be utilized in extending the longevity of a feeding tube. An example of a laser process is laser ablation. One example of a laser is a Kr-F excimer laser (248 nm). A method of utilizing this laser is described by Suggs AE (Kr-F laser surface treatment of poly(methyl methacrylate, glycol-modified poly (ethylene terephthalate), and polytetrafluoroethylene for enhanced adhesion of escherichia coli K-12 Suggs AE. 2002. Master of Science Thesis, Materials Science and Engineering, (Virginia Polytechnic Institute and State University).

Following laser treatment of a feeding tube, biofilm formation and proliferation will be inhibited or delayed.

It should be evident to those skilled in the art that laser processes vary considerably. Therefore, the present invention is not limited to this one particular variation of a laser process.

### EXAMPLE 10

Adding One or more Anti-biofllm Mechanisms via Plasma Processes

Various plasma processes can be used to surface treat a feeding tube. Plasma processes involve a plasma reaction that either results in modification of the molecular structure of the feeding tube or atomic substitution. Such processes include but are not limited to plasma sputtering and etching, plasma implantation, plasma deposition, plasma polymerization, laser plasma deposition, plasma spraying, and so forth. A reactive plasma etching process, such as that described in described in Lee et al U.S. Pat. No. 6,033,582, incorporated herein by reference in its entirety, can be employed to modify the surface of a feeding tube such that the resulting roughness, porosity and texture are optimized for application of an anti-biofilm mechanism.

It should be evident to those skilled in the art that plasma processes vary considerably. Therefore, the present invention is not limited to this one particular variation of a plasma process.

### EXAMPLE 11

Adding One or more Anti-biofilm Mechanisms via Chemical Plating

Chemical plating can be used to surface treat a feeding tube. It involves the formation of a thin adherent layer of a chemical on a feeding tube. One example of a chemical is a metal. When a metal is used in plating the feeding tube the process is referred to as electroplating. Preferred metals include Ti, Au, Al and Si, and the metal elements from the following groups of the periodic table: IIIB, IVB, VB, VIB, VIIB, VIIIB, IB, IIB, IIA, IVA, and VA (excluding As) in the periods 4, 5 and 6, (see Periodic Table as published in Merck Index 10th Ed., 1983, Merck and Co. Inc., Rahway, N.J., Martha Windholz). Other metals could include elements from the groups one through sixteen of the periodic table. As described in U.S. Pat. No. 6,267,782, incorporated herein by reference in its entirety, various methods can be used to associate antimicrobial metal with medical articles. Such methods can be used to apply antimicrobial metals to feeding tubes.

It should be evident to those skilled in the art that chemical plating processes vary considerably. Therefore, the present invention is not limited to this one particular variation of chemical plating.

### EXAMPLE 12

Adding One or more Anti-biofilm Mechanisms via Grafting

Grafting, or graft polymerization, can also be used to surface treat a feeding tube. This method involves the creation of free radicals on a feeding tube surface. These free radicals are able to initiate copolymerization with available monomers, or reactive oligomers, thereby generating a graft polymer layers. Anti-biofilm mechanism can be entrapped within graft layers.

A grafting process, such as that described in described in U.S. Pat. Application 2002/0133072, incorporated herein by reference in its entirety, can be employed to modify the surface of a feeding tube such that an anti-biofilm mechanism can be entrapped within graft layers.

It should be evident to those skilled in the art that grafting processes vary considerably. Therefore, the present invention is not limited to this one particular variation of a grafting process.

### EXAMPLE 13

Adding One or more Anti-biofilm Mechanisms via Bombardment with Energetic Particles (Plasma Immersion or Ion Implantation)

Ion implantation is another method of surface treating a feeding tube. It involves the bombardment of a surface with high-energy non-metal, metal and/or semi-metal ions to yield a thin, wear and corrosion-resistant protective layer.

It should be evident to those skilled in the art that methods of bombardment with energetic particles (plasma immersion or ion implantation) vary considerably. Therefore, the present invention is not limited to this one particular variation of bombardment with energetic particles (plasma immersion or ion implantation)

### EXAMPLE 14

Adding One or more Anti-biofilm Mechanisms via Gamma Radiation

Gamma radiation is another method of surface treating a feeding tube. Gamma ray treatments can be used for cross-linking of feeding tube polymer coatings and/or formation of thin polymeric films on a feeding tube surface.

With gamma radiation, new functional groups can be introduced onto a feeding tube surface. The newly created functional groups may possess intrinsic antimicrobial activity, thus extending the longevity of the feeding tube. In this process, antimicrobial substances may also be linked covalently to the functional surface groups.

A gamma radiation process, such as that described in described in U.S. Pat. Application 2002/0037944, incorporated herein by reference in its entirety, can be employed to modify the surface of a feeding tube.

It should be evident to those skilled in the art that gamma radiation processes vary considerably. Therefore, the present invention is not limited to this one particular variation of a gamma radiation process.

### EXAMPLE 15

Adding One or more Anti-biofilm Mechanisms via Glow Discharge

Glow discharge, or corona discharge, is another method of surface treating a feeding tube. It also introduces new functional groups on the feeding tube surface. The newly created functional groups may possess intrinsic antimicrobial activity thus extending the longevity of the feeding tube. In this process, antimicrobial substances may also be linked covalently to the functional surface groups.

Such a glow discharge method is described in Karwoski et al U.S. Pat. No. 4,632,842, incorporated herein by reference in its entirety.

It should be evident to those skilled in the art that glow discharge processes vary considerably. Therefore, the present invention is not limited to this one particular variation of a glow discharge process.

### EXAMPLE 16

Adding One or more Anti-biofilm Mechanisms via Formation of a Drug-polymer Conjugate

Forming a drug-polymer conjugate is another method of surface treating a feeding tube.

It involves the covalent attachment of a therapeutic agent such as a drug to the feeding tube polymer. Prior to polymerization, covalent linkage of an agent to a monomer occurs.

An example of this process is used in the coronary stent industry where stents are modified to have antithrombogenic and antibacterial activity by covalent attachment of heparin to silicone with subsequent entrapment of antibiotics in cross-linked collagen bound to the heparinized surface. This process is described in Fallgren C, Utt M, Petersson A C, Ljungh A, Wadstrom T. In vitro anti-staphylococcal activity of heparinized biomaterials bonded with combinations of rifampicin. Zent Fur Bakt-Int J Med Micro Vir Paraotol Infect Dis 1998;287(1-2):19-31.

Selection of therapeutic agents is dependant on compatible chemistry with the synthetic reaction scheme utilized in the preparation of the feeding tube.

It should be evident to those skilled in the art that processes involving the formation of a drug-polymer conjugate vary considerably. Therefore, the present invention is not limited to this one particular variation of forming a drug-polymer conjugate.

### EXAMPLE 17

Adding One or more Anti-biofilm Mechanisms via a Biomimetic Process

A biomimetic surface can be applied to a feeding tube. Biomimetic surfaces mimic the body's natural defense by exuding a substance to a surface that is subsequently shed and replenished. In the shedding process, attached biofilm is released from the feeding tube. This mimics the body's natural shedding of tissue cells and mucus. This technology relies on higher-molecular-weight polysilanes as cross-linking agents for silicones. Therapeutic agents can also be delivered to the device surface for site-specific activity.

A biomimetic process, such as that described in described in Gorman et al WO02090436 and Gorman et al WO0134695, incorporated herein by reference in its entirety, can be employed to modify the surface of a feeding tube.

It should be evident to those skilled in the art that biomimetic processes vary considerably. Therefore, the present invention is not limited to this one particular variation of a biomimetic process.

### EXAMPLE 18

Adding One or more Anti-biofilm Mechanisms via Formation of a Hydrophilic Surface or a Hydrophobic Surface

Surface treatments of a feeding tube can generate a hydrophilic surface or a hydrophobic surface. Since it has already been established that there is a positive correlation between some hydrophobic surfaces and biofilm formation, preparation of hydrophilic coatings provide another method of inhibiting and/or delaying the formation and/or proliferation of fungal and/or bacterial biofilm.

Such an anti-biofilm mechanism feeding tube can be achieved by forming a hydrophilic surface or a hydrophobic surface. Such a method is described in Price et al (Price C, Waters M G J, Williams D W, Lewis M A O, Stickler D. Surface modification of an experimental silicone rubber aimed at reducing initial candidal adhesion. J Biomed Mater Res (Appl Biomater) 2002; 63: 122-128), incorporated herein by reference in its entirety.

It should be evident to those skilled in the art that processes involving the formation of a hydrophilic surface or a hydrophobic surface vary considerably. Therefore, the present invention is not limited to this one particular variation of forming a hydrophilic surface or a hydrophobic surface.

### EXAMPLE 19

Adding One or more Anti-biofilm Mechanisms via a Diffusion Process

Diffusion processes are another method of surface treating a feeding tube. Nitriding is one example of a diffusion process that can be used to surface treat a feeding tube. In nitriding, hard and wear resistant layers are generated by nitrogen or nitrogen and carbon diffusion into the bulk material. Carburizing is another example of a diffusion process that can be used to surface treat a feeding tube.

A diffusion process, such as that described in described in Davidson et al U.S. Pat. No. 5,647,858, incorporated herein by reference in its entirety, can be employed to modify the surface of a feeding tube.

It should be evident to those skilled in the art that diffusion processes vary considerably. Therefore, the present invention is not limited to this one particular variation of a diffusion process.

### EXAMPLE 20

Adding One or more Anti-biofilm Mechanisms via a Flame Treatment Process

Flame treatment is another method of surface treating a feeding tube. This method introduces oxygen-containing polar groups onto a feeding tube surface. The presence of such groups on the feeding tubes leads to enhanced adhesion of an anti-biofilm mechanism.

A flame treatment process, such as that described in described in Ishihara et al U.S. Pat. No. 6,159,651, incorporated herein by reference in its entirety, can be employed to modify the surface of a feeding tube.

It should be evident to those skilled in the art that flame treatment processes vary considerably. Therefore, the present invention is not limited to this one particular variation of a flame treatment process.

### EXAMPLE 21

Adding One or more Anti-biofilm Mechanisms via an Ultraviolet (UV) Process

Another method of surface treating a feeding tube involves an ultraviolet process, An ultraviolet (UV) process employs photons, usually having low wavelength and high energy, which are used to activate a variety of chemical reactions.

An ultraviolet process, such as that described in described in Ishihara et al U.S. Pat. No. 6,159,651, incorporated herein by reference in its entirety, can be employed to modify the surface of a feeding tube.

It should be evident to those skilled in the art that ultraviolet (UV) processes vary considerably. Therefore, the present invention is not limited to this one particular variation of an ultraviolet (UV) process.

### EXAMPLE 22

Adding One or more Anti-biofilm Mechanisms via Surface Functionalization

An anti-biofilm mechanism feeding tube can also be achieved by a surface functionalization method. Such a surface functionalization method is described in Everaert E P et al (Everaeart E P, Mahieu H F, van de Belt-Gritter B, Peeters A J, Verkerke G J, van der Mei H C, Busscher H J. Biofilm formation in vivo on perfluoro-alkylsiloxane-modified voice prosthesis. Arch Otolaryngol Head Neck Surg. 1999 December;125(12):1329-32) incorporated herein by reference in its entirety.

It should be evident to those skilled in the art that surface functionalization processes vary considerably. Therefore, the present invention is not limited to this one particular variation of a surface functionalization process.

Placement of an Extended-longevity Feeding Tube

Placement of an Extended-longevity Feeding Tube via Percutaneous Endoscopic Gastrostomy (PEG)

Despite some existing variation with respect to the components of the professional team and technique, the safest approach is an experienced professional team consisting of a surgeon, an anesthesiologist, an endoscopist and a G.I. nurse endoscopic technician. After it is determined a patient has met the guidelines of the ASGE, informed consent is obtained from the patient, nearest of kin, guardian or power of attorney. However, in a few cases, there is no guardian or power of attorney to give informed consent. In these instances, three physicians review the case and deem that a PEG is necessary for the health of the patient.

Following this, the fasting patient is taken to the endoscopic suite. The usual monitoring devices are put in place; i.e., blood pressure, respiration, pulse oximetry and EKG. A crash cart with resuscitation equipment is also readily available and present in the suite. The anesthesiologist administers conscious sedation or monitored anesthesia care (MAC) intravenously. This usually consists of a drug such as midazolam HCl, fentanyl or propofol (2,6-diisopropyl phenol). Nasal oxygen is administered to all patients. After local anesthesia such as Hurricane® is administered to the nasopharynx, a bite block is placed. The endoscopist, in general, introduces a video fiberscope such as the Olympus GIF 100 Video Fiberscope into the stomach. The stomach is insufflated with air. When the scope is in the proper position a light is usually visible on the exterior skin overlying the upper epigastrium. The surgeon applies pressure on the abdominal wall in the area of the light. An indentation in the wall of the stomach is clearly visible by the endoscopist. After antiseptic is applied to the skin, the surgeon makes a small incision and introduces a trochar. The trochar is then visualized by the endoscopist. Then, a plastic 20 french tube is inserted through the trochar. A wire is then introduced through the tube into the stomach. This wire is snared by the endoscopist, and the wire and endoscope are removed. The wire is now protruding through the mouth and is attached to a similar wire to which the extended-longevity gastrostomy tube with a mushroom bulb are attached. This is pulled through the esophagus through the plastic tube opening in the stomach and fits snuggly against the interior wall of the stomach. The protruding extended-longevity feeding tube is anchored to the skin with a plastic crossbar. The endoscopist then repeats an upper G. I. endoscopy procedure to ensure that the mushroom bulb is in the proper location, and that there are no complications such as bleeding or blanching of the mucosa. As an infection prophylaxis measure, the skin surrounding the PEG site is covered with an antibiotic such as bacitracin and a bandage. Monitoring is continued until the patient is awake.

An extended-longevity feeding tube can be secured with an external bolster, crossbar, or other device to secure the tube against the skin overlying the abdominal wall. If necessitated, the extended-longevity feeding tube can be secured in a specific position using tape. Bandages over the extended-longevity feeding tube are not needed.

It should be recognized that while this is one method of placing an extended-longevity feeding tube, there may be other variations with respect to the method of placing an extended-longevity feeding tube. The push method and introducer method are examples of such variations. Placement of an extended-longevity feeding tube can also occur via jejunal extension through a PEG (PEG-J), direct endoscopic jejunostomy (D-PEG), radiological approaches, open surgical gastrostomy or laparoscopic gastrostomy, and a transnasal approach.

Use of an Extended-longevity Feeding Tube

Administering Feedings Using an Extended-longevity Feeding Tube

Once the extended-longevity feeding tube is in place, at least four hours should elapse before extended-longevity tube feedings can be initiated. During this time the patient is kept NPO (no food by mouth) and on intravenous fluids. The patient typically can be fed by a choice of three methods of enteral nutrition: delivery by means of bolus feeding, continuous pump feeding, or gravity feeding. Bolus feeding involves the intermittent infusion of blenderized food or formula through the extended-longevity feeding tube. A feeding pump is a piece of mechanical equipment that pumps blenderized foods or formulas in a continuous uninterrupted manner. Gravity feeding involves hanging or holding a bag of blenderized food or formula. This method uses the force of gravity to deliver the blenderized food or formula to the stomach via the extended-longevity feeding tube. The extended-longevity feeding tube does not limit the choice of feeding.

In the adult patient, the extended-longevity feeding tube generally protrudes ten to fifteen inches from the skin overlying the abdominal wall. Attached to the extended-longevity feeding tube typically is an adapter piece that with a plug cap or a flip cap whose function is to seal off the tube when the patient is not being fed. Biofilm does not pose a threat to the distal portion of the extended-longevity feeding tube. Therefore, the present invention does not necessitate that the tubing be surface treated between the adapter piece and the enteral feeding pump nor between the enteral feeding pump and the enteral feeding container, although the entire length of tubing may be surface treated if desired.

Example of nutritional fluids that can be utilized during administration of feedings include EnsurePlus®, FiberSource®, Jevity®, Osomolite®, or similar fluids. One example of a feeding regimen involves the patient receiving continuous infusions of approximately 1,500 mL per day via six bolus feedings of 250 mL for a total of 1500 mL per day.

Enteral feeding formulas can be prepared, powdered, or blenderized. The formula should be at room temperature at the time of administration.

Pre-feeding Checking of an Extended-longevity Feeding Tube

Before enteral feedings can be administered to the patient via an extended-longevity feeding tube, the caregiver should first check it. This generally involves several steps. Prior to checking the extended- feeding tube, the caregiver should wash his or her hands. The extended-longevity feeding tube should first be checked to ensure that it has not deviated from its position at the time of placement. Using a ruler, this can be accomplished by measuring from the stoma to the distal end of the extended-longevity feeding tube. Next, the extended-longevity feeding tube should be checked to ensure that it is not clogged from the previous feeding. This can be accomplished by drawing a syringe with approximately five to ten milliliters of water for adult patients or three to five milliliters for pediatric patients. Next, the plug or cap at the distal end of the extended-longevity feeding tube is opened. With one hand, a stethoscope is placed in the left lower quadrant of the abdomen, just superior to the iliac crest. With the other hand, the syringe is placed in the extended-longevity feeding tube and the plunger is depressed. Then, using the stethoscope, the caregiver auscultates for a gurgling or a “whooshing” sound. If this sound is not auscultated, then this procedure should be repeated. If the sound is still not auscultated, then no feedings should be administered to the patient until the extended-longevity feeding tube is assessed by a physician. Finally, the gastric contents should be aspirated from the coated feeding tube and measured for residual from the previous feeding.

If the patient is being fed continuously, the above steps should be repeated approximately every four to eight hours. Using a fifty milliliter bulbed or piston syringe, gastric contents are gently aspirated. If the amount aspirated through the extended-longevity feeding tube is more than an amount pre-determined by the physician, then this procedure should be repeated once again in approximately thirty to sixty minutes. If the amount of residual aspirated is still excessive, then no feedings should be administered to the patient via the extended-longevity feeding tube until the problem is assessed by a physician. In either case, the amount of residual fluid withdrawn should be reinstilled into the extended-longevity feeding tube. This is done to ensure that the patient is not deprived of essential nutrients. An excessive amount of residual fluid is usually indicative of delayed gastric emptying.

Conversely, the amount of residual fluid aspirated from the extended-longevity feeding tube by the syringe could be less than that pre-determined by the physician. This is usually a sign that the patient's stomach is empty. If this scenario should occur, then the residual fluid should be injected back into the coated feeding tube. Following this, approximately twenty-five to fifty milliliters of water for the adult patient or fifteen to thirty milliliters of water for the pediatric patient should be drawn into the syringe and injected into the extended-longevity feeding tube.

Position of Patient Prior to Feeding with an Extended-longevity Feeding Tube

Patients should remain in an upright position while receiving feedings from the extended-longevity feeding tube. They should also maintain this position for approximately sixty minutes after feeding has ceased.

Administering Medications (if Prescribed) Using an Extended-longevity Feeding Tube

The aforementioned bolus feeding method can be used to deliver medications via the extended-longevity feeding tube. Liquid medications can be administered via the extended-longevity feeding tube. Solid tablets can also be administered via the extended-longevity feeding tube. However, they should first be crushed and dissolved in water before being administered via the extended-longevity feeding tube.

Gastric Decompression (if Prescribed) Using an Extended-longevity Feeding Tube

To perform gastric decompression of an extended-longevity feeding tube, the adaptor of the feeding tube is first removed. Then, the tube is allowed to drain into a collecting bag or basin.

Removal of an Extended-longevity Feeding Tube

An extended-longevity feeding tube can be removed. First, the operator grasps the tube near the skin line. Then, the operator places the other hand around the stoma site. Finally the operator pulls upward using the hand grasping the tube.

Another method of removing an extended-longevity feeding tube involves cutting the tube at skin level and removing the remaining tube endoscopically.

Maintenance of an Extended-longevity Feeding Tube

The stoma and exterior surface of an extended-longevity feeding tube can be cleaned using soap, water, and cotton swabs.

Post-feeding Checking of an Extended-longevity Feeding Tube

Following administration of feeding and/or medication, the extended-longevity feeding tube should be flushed with approximately 100 mL. Every four hours thereafter, the extended-longevity tube is checked for residuals and is flushed with 100 mL of water.

It should be recognized that while this is one preferred method of using an extended-longevity tube, there may be local variations with respect to its use.

Clinical Observation

Replacement feeding tubes that are placed through existing stoma last longer than those tubes which are placed initially. Since the latter procedure involves traversing the oropharyngel canal and esophagus and the former does not, this lends support to the theory of colonization during the time of initial feeding tube placement. Implications with respect to the present invention are that the concentration of therapeutic agent and duration of therapeutic agent elution would not have to be on a large order of magnitude in order to achieve an extended-longevity gastrostomy tube.

Candida albicans is known to form biofilm on other medical devices and limit their longevity. Consequently, elements of the present invention may also be applicable to the following medical devices: artificial voice prosthesis, central venous catheters, intrauterine devices, mechanical heart valves, breast implants, penile prosthesis, axillo-femoral vascular, prosthetic hip, knee, and/or shoulder joints, prosthetic palates, dentures, and urinary catheters.

The foregoing detailed description of the invention has been presented for purposes of illustration and description. It is not intended to be exhaustive or to limit the invention to the precise form disclosed. Many modifications and variations are possible in light of the above teaching. The described embodiments were chosen in order to best explain the principles of the invention and its practical application to thereby enable others skilled in the art to best utilize the invention in various embodiments and with various modifications as are suited to the particular use contemplated. It is intended that the scope of the invention be defined by the claims appended hereto.

What is claimed is:

1. A feeding apparatus, comprising:
a feeding tube, said feeding tube including one or more surfaces having one or more anti-biofilm mechanisms.

2. The feeding apparatus of claim 1, wherein:
said anti-biofilm mechanism inhibits or delays the formation and/or proliferation of fungal and/or bacterial biofilm.

3. The feeding apparatus of claim 1, wherein:
said anti-biofilm mechanism blocks steps of a biofilm lifeycle.

4. The feeding apparatus of claim 1, wherein:
said anti-biofilm mechanism blocks or disrupts fungal and/or bacterial arrangement and/or attachment.

5. The feeding apparatus of claim 1, wherein:
said anti-biofilm mechanism interferes with fungal and/or bacterial extracellular matrix formation.

6. The feeding apparatus of claim 1, wherein:
said anti-biofilm mechanism delivers signal blockers to threatened areas to abort fungal and/or bacterial biofilm formation.

7. The feeding apparatus of claim 1, wherein:
said anti-biofilm mechanism undermines the varied survival strategies of biofilm cells.

8. The feeding apparatus of claim 1, wherein:
said anti-biofilm mechanism induces fungal and bacterial cells to detach, then targets them with an anti-biofilm mechanism.

9. The feeding apparatus of claim 1, wherein:
said anti-biofilm mechanism is of an antifungal drug class.

10. The feeding apparatus of claim 1, wherein:
said anti-biofilm mechanism is of an echinocandin antifungal drug class.

11. The feeding apparatus of claim 1, wherein:
said anti-biofilm mechanism is caspofungin, micafungin, or anidulafungin of an echinocandin antifungal drug class.

12. The feeding apparatus of claim 1, wherein:
said anti-biofilm mechanism is a lipid formulation of amphotericin B (liposomal amphotericin B and/or amphotericin B lipid complex).

13. The feeding apparatus of claim 1, wherein:
said anti-biofilm mechanism is of the antibacterial drug class.

14. The feeding apparatus of claim 1, wherein:
said anti-biofilm mechanism is a metal, metallic ion, metal alloy, or metal conjugated with another anti-biofilm mechanism.

15. The feeding apparatus of claim 1, wherein:
said anti-biofilm mechanism is an antiseptic.

16. The feeding apparatus of claim 1, wherein:
said anti-biofilm mechanism is a disinfectant.

17. The feeding apparatus of claim 1, wherein:
said anti-biofilm mechanism blocks gene expression.

18. The feeding apparatus of claim 1, wherein:
said anti-biofilm mechanism inhibits and/or delays formation and/or proliferation of granulation tissue.

19. The feeding apparatus of claim 1, wherein:
said anti-biofilm mechanism inhibits and/or delays deposition and/or proliferation of inorganic salts.

20. A method of preparing an extended-longevity feeding tube, comprising the step of:
adding one or more anti-biofilm mechanisms to one or more surfaces of a feeding tube.

21. The method of claim 20, wherein:
said step of adding one or more anti-biofilm mechanisms includes formation of covalent drug tether from which one or more anti-biofilm mechanisms can be lysed.

22. The method of claim 20, wherein:
said step of adding one or more anti-biofilm mechanisms includes formation of a polymeric sheet.

23. The method of claim 20, wherein:
said step of adding one or more anti-biofilm mechanisms includes a bonding process.

24. The method of claim 20, wherein:
said step of adding one or more anti-biofilm mechanisms includes a vacuum deposition or vacuum coating process.

25. The method of claim 20, wherein:
said step of adding one or more anti-biofilm mechanisms includes a hydrogel encapsulation process.

26. The method of claim 20, wherein:
said step of adding one or more anti-biofilm mechanisms includes a solvent casting process.

27. The method of claim 20, wherein:
said step of adding one or more anti-biofilm mechanisms includes a dip coating process.

28. The method of claim 20, wherein:
said step of adding one or more anti-biofilm mechanisms includes a spray coating process.

29. The method of claim 20, wherein:
said step of adding one or more anti-biofilm mechanisms includes a laser process.

30. The method of claim 20, wherein:
said step of adding one or more anti-biofilm mechanisms includes a plasma process.

31. The method of claim 20, wherein:
said step of adding one or more anti-biofilm mechanisms includes a chemical plating process.

32. The method of claim 20, wherein:
said step of adding one or more anti-biofilm mechanisms includes a grafting process.

33. The method of claim 20, wherein:
said step of adding one or more anti-biofilm mechanisms includes a process of energetic particle bombardment including plasma immersion, ion implantation, ion beam treatments, or electron beam treatments.

34. The method of claim 20, wherein:
said step of adding one or more anti-biofilm mechanisms includes a gamma radiation process or gamma ray process.

35. The method of claim 20, wherein:
said step of adding one or more anti-biofilm mechanisms includes a corona or glow discharge process.

36. The method of claim 20, wherein:
said step of adding one or more anti-biofilm mechanisms includes a biomimetic process.

37. The method of claim 20, wherein:
said step of adding one or more anti-biofilm mechanisms includes the formation of a hydrophilic or hydrophobic surface.

38. The method of claim 20, wherein:
said step of adding one or more anti-biofilm mechanisms includes formation of a drug-polymer conjugate.

39. The method of claim 20, wherein:
said step of adding one or more anti-biofilm mechanisms includes a diffusion process including nitriding or carburizing.

40. The method of claim 20, wherein:
said step of adding one or more anti-biofilm mechanisms includes a flame treatment processes.

41. The method of claim 20, wherein:
said step of adding one or more anti-biofilm mechanisms includes an ultraviolet (UV) process.

42. A feeding apparatus, comprising:
a feeding tube, said feeding tube includes one or more reservoirs, said reservoirs include one or more anti-biofilm mechanisms.

43. A feeding apparatus, comprising:
a feeding tube, said feeing tube includes one or more surfaces having a constituent polymer matrix, said constituent polymer matrix including one or more anti-biofim mechanisms.

44. A method of placing an extended-longevity feeding tube, comprising the steps of:
creating an opening in a patient; and
inserting a feeding tube in said patient, said feeding tube including one or more surfaces having one or more anti-biofilm mechanisms.

45. The method of claim 44, wherein:
said steps of creating and inserting are part of a percutaneous endoscopic gastrostomy (PEG).

46. The method of claim 44, wherein:
said steps of creating and inserting are part of a jejunal extension through a PEG (PEG-J).

47. The method of claim 44, wherein:
said steps of creating and inserting are part of a direct endoscopic jejunostomy (D-PEJ).

48. The method of claim 44, wherein:
said steps of creating and inserting are part of a radiological process.

49. The method of claim 44, wherein:
said steps of creating and inserting are part of a surgical process or laparoscopic process.

50. The method of claim 44, wherein:
said steps of creating and inserting are part of a transnasal process.

51. A method for using an extended-longevity feeding tube, comprising:
installing a feeding tube in a patient, said feeding tube including one or more surfaces having one or more anti-biofilm mechanisms; and
feeding said patient using said feeding tube.

52. The method of claim 51, wherein:
said step of feeding includes administration of bolus feedings.

53. The method of claim 51, wherein:
said step of feeding includes administration of continuous feedings.

54. The method of claim 51, wherein:
said step of feeding includes administration of gravity feedings.

55. The method of claim 51, wherein:
said step of feeding includes pre-feeding checking.

56. The method of claim 51, wherein:
said step of feeding includes post-feeding checking.

57. The method of claim 51, further comprising the step of:
maintaining said feeding tube.

58. The method of claim 51, wherein:
said step of feeding includes administering one or more medications.

59. The method of claim 51, further comprising the step of:
performing gastric decompression.

60. The method of claim 51, further comprising the step of:
removing said feeding tube.
